(19) United States
(12) Patent Application Publication
(10) Pub. No.: US 2020/0360676 A1
(43) Pub. Date: Nov. 19, 2020

(54) Patient Mittens

(71) Applicant: Cassandra Bunn, Glen Allen, VA (US)

(72) Inventor: Cassandra Bunn, Glen Allen, VA (US)

(21) Appl. No.: 16/985,135

(22) Filed: Aug. 4, 2020

Related U.S. Application Data
(63) Continuation-in-part of application No. 15/889,108, filed on Feb. 5, 2018.
(60) Provisional application No. 62/882,824, filed on Aug. 5, 2019.

(51) Int. Cl. A61M 35/00; A41D 19/01; A41D 19/00

(52) U.S. Cl. CPC A61M 35/10; A41D 2400/32; A41D 19/0024; A41D 19/01

(57) ABSTRACT

Various embodiments of patient-mittens are disclosed and described. Such embodiments may include a hand-covering portion attached to a hollow elongate-sleeve portion. The hand-covering portion may cover the hand and fingers, preventing the patient from scratching themselves. The elongate-sleeve portion may cover a majority of an arm. Opposite from the hand-cover portion, there may be an arm-grabber attached at an end of the elongate-sleeve. This arm-grabber may squeeze the upper arm, above the elbow, to help keep the patient-mitten from coming off. Some embodiments, may include an access-slit on the elongate-sleeve for placing IVs and the like in a patient wearing the patient-mitten. The patient-mitten may help to minimize IVs and vital sign sensors from becoming dissociated from the patient. Some embodiments may be UV permeable to encourage vitamin D production in the patient. Some embodiments may provide a conducive environment for treating skin conditions on the arm, hand, or fingers.

### PRIORITY NOTICE

The present application claims priority under 35 U.S.C. § 119(e) to U.S. Provisional Patent Application Ser. No. 62/882,824 filed on Aug. 5, 2019, the disclosure of which is incorporated herein by reference in its entirety.

The present patent application is a continuation-in-part (CIP) of U.S. non-provisional patent application Ser. No. 15/889,108 filed on Feb. 5, 2018, and claims priority to said U.S. non-provisional patent application under 35 U.S.C. § 120. The above-identified U.S. non-provisional patent application is incorporated herein by reference in its entirety as if fully set forth below.

### TECHNICAL FIELD OF THE INVENTION

The present invention relates in general to mittens (e.g., hand coverings) attached to arm sleeves (e.g., arm coverings) and more specifically to such mittens for use with patients with certain medical needs.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Newborn human babies, infants, and prematurely born babies (preemies) often have fingernails of sufficient length to cause scratching injuries to themselves and are typically not yet able to control themselves sufficiently to not scratch themselves and cause injury to themselves. Thus, there is a need for protective mittens for this class of people. And the art has responded by providing protective mittens. However, most of those protective mittens suffered from being very susceptible to falling off, because the sleeve portion, if any, was not long enough and the arm grabbing portion, if any, of some such mittens was squeezing the arm below the elbow.

The same self-harming problems may be true for larger children, youths, adolescents, and adults who may be confused, have diminished mental capacity, and/or that may have certain conditions that would cause such people to scratch, grab, or pick at their skin, eyes, noses, and the like, or that could cause harm to themselves. Also, those with certain muscle and/or skeletal issues (such as, but not limited to, contracture) may have curled fingers where their nails can dig into their hand/flesh. In these cases, a longer sleeve may keep things such as, wires (e.g., wires to sensors), tubes (e.g., IV and/or feeding tubes), itchy spots, and/or other areas of focus out of mind of the given patient; and thus, reduce undesirable picking, grabbing, and scratching.

Preemies have additional protective mitten needs above and beyond the need to protect from self-scratching. Because of their premature birth, preemies spend significant amounts of time in incubators and attached to various medical device equipment, such as, but not limited to, vital sign monitoring devices, IVs, feeding tubing, ventilators, and the like. Because of natural movements of the preemie, such medical device equipment routinely becomes dissociated (e.g., disconnected) from the preemie, requiring re-attachment, causing undesirable discomfort and pain in the preemie. Thus, it would be desirable to minimize such medical device equipment from becoming dissociated from the preemie. And an appropriate patient mitten could satisfy this need by making it harder for the preemie to inadvertently dissociate such attached medical device equipment. The appropriate patient mitten could cover and protect the points of attachment to the preemie of such medical device equipment.

However, preemies also need sufficient access to UV (ultraviolet) light for the production of vitamin D and so covering the arms of the preemie with a sleeve of the patient mitten may create an unexpected additional problem. And so there may be a need for a UV permeable patient mitten, that allows sufficient UV light to reach the preemie, but still functions to minimize dissociation of the various attached medical device equipment, may also still function to protect the preemie from self-inflicted scratches.

Various skin problems (e.g., patches of dry skin, rashes, eczema, and the like) are also very common with newborn human babies, infants, preemies, and toddlers. Presently, care givers apply various creams, lotions, salves, ointments, and medicaments to the affected skin areas. However, when the affected skin area may be the arms, hands, and/or fingers, treating such areas can be problematic as these areas are prone to having the applied treatment lost to adsorption into clothing or lost due to interactions in the environment. It would be desirable if such affected skin areas, after treatment, could then be covered by an appropriate patient mitten that would prevent the applied treatment being lost to the environment or adsorbed into clothing.

The same or similar skin problems of the arms, hands, and/or fingers may be true for older children, youth, and adults where such people may undesirably scratch, grab, pull, and/or pick at such irritated regions of skin. For example, people suffering from dementia, Alzheimer's, shingles, OCD, sensory issues, PTSD, liver problems, and others may have irritated skin of the arms, hands, and/or fingers.

There is a need in the art for various patient mittens to address the above identified problems, such as, but not limited to, to prevent self-inflicted scratching, to prevent self-damage, to prevent fingers from curling down in an undesired fashion, to help keep a hand open, to minimize dissociation of attached medical device equipment, to permit vitamin D production, and/or to protect treated skin from losing the treatment from the treated skin.

It is to these ends that the present invention has been developed.

### BRIEF SUMMARY OF THE INVENTION

To minimize the limitations in the prior art, and to minimize other limitations that will be apparent upon reading and understanding the present specification, the present invention describes various embodiments of patient-mittens. Some embodiments of patient-mittens may comprise a hand-cover attached to a hollow elongate-sleeve. The hand-cover may cover the hand and fingers, preventing the patient from scratching themselves (and in some embodiments, does not include finger separators). The elongate-sleeve may cover a majority of an arm of the patient. Opposite from the hand-cover, there may be an arm-grabber attached at an end of the elongate-sleeve. This arm-grabber may squeeze the upper arm, above the elbow, to help keep the patient-mitten from coming off (or in some embodiments, the arm-grabber may grab the arm at or below the elbow). Some embodiments of patient-mittens may include an access-slit on the elongate-sleeve for placing medical tubing (e.g., IVs), vital sign monitoring sensors, and the like in a patient wearing the patient-mitten. The patient-mitten may help to minimize the attached medical tubing and vital sign sensors from becoming dissociated from the patient. Some embodiments of patient-mittens may be UV permeable to encourage vitamin D production in the patient. Some embodiments of patient-mittens may provide a conducive environment for treating skin conditions on the arm, hand, or fingers, that the worn patient-mitten may then cover and protect.

It is an objective of the present invention to provide a patient mitten to prevent (or minimize) self-inflicted scratching and/or to prevent (or minimize) self-damage.

It is another objective of the present invention to provide a patient mitten that may provide warmth and/or comfort to the patient (wearer).

It is another objective of the present invention to provide a patient mitten that covers and protects various medical device equipment attached to a patient's arm, hand, and/or fingers from becoming inadvertently dissociated from the patient's body.

It is another objective of the present invention to train patient behavior in avoiding undesirable picking, scratching, pulling, grabbing, and/or the like of the of any medical devices attached to the patient's arms, hands, and/or fingers.

It is another objective of the present invention to train patient behavior in avoiding undesirable picking, scratching, pulling, grabbing, and/or the like of the patient's skin of the patient's arm, hand, and/or fingers.

It is another objective of the present invention to provide a patient mitten that covers and protects various medical device equipment attached to a patient's arm, hand, and/or fingers from becoming inadvertently dissociated, but while also providing a means for the care giver to access such attached medical device equipment, without having to remove the patient mitten.

It is another objective of the present invention to provide a patient mitten that may be at least be partially UV permeable, to facilitate vitamin D production in the patient.

It is another objective of the present invention to provide a patient mitten that may be at least partially liquid impermeable, e.g., water resistant or waterproof.

It is another objective of the present invention to provide a patient mitten that may be at least be partially liquid impermeable, e.g., water resistant or waterproof; but that is also at least partially air permeable, to provide for “breathing” and/or ventilation.

It is another objective of the present invention to provide a patient mitten that may be at least partially soft and/or flexible.

It is another objective of the present invention to provide a segment of horizontal elastic band in the hand-covering of a given mitten to facilitate flipping the mitten open or closed.

It is yet another objective of the present invention to provide a patient mitten that may protect treated skin beneath the worn patient mitten.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1A may depict an embodiment of a patient-mitten, shown from a top (dorsal) or from a bottom (ventral) view, with respect to a patient's arm.

FIG. 1B may depict an embodiment of a patient-mitten, shown from a top (dorsal) or from a bottom (ventral) view, with respect to a patient's arm.

FIG. 1C may depict an elongate-sleeve portion of a patient-mitten, depicting an access-slit and slit-fastener, shown from a top (dorsal) or from a bottom (ventral) view, with respect to a patient's arm.

FIG. 1D may depict an elongate-sleeve portion of a patient-mitten, depicting an access-slit and slit-fastener, shown from a top (dorsal) or from a bottom (ventral) view, with respect to a patient's arm.

FIG. 1E may depict a partial view of a patient-mitten, shown from a top (dorsal) or from a bottom (ventral) view, with respect to a patient's arm, showing a section of medical tubing emerging from an access-slit in an elongate-sleeve of that patient-mitten.

FIG. 1F may depict a partial view of a patient-mitten, shown from a top (dorsal) or from a bottom (ventral) view, with respect to a patient's arm, showing a section of medical tubing emerging from an access-slit in an elongate-sleeve of that patient-mitten.

FIG. 2 may depict an embodiment of a mitten-with-attachment-anchors, shown from a top (dorsal) or from a bottom (ventral) view, with respect to a patient's arm, wherein at least one attachment-anchor may be shown attached to an elongate-sleeve of the mitten-with-attachment-anchors.

FIG. 3 may depict an embodiment of a mitten-with-pocket, shown from a top (dorsal) or from a bottom (ventral) view, with respect to a patient's arm, wherein at least one pocket may be shown attached to an elongate-sleeve of the mitten-with-pocket.

FIG. 4A may depict an embodiment of a UV-permeable-mitten, shown from a top (dorsal) or from a bottom (ventral) view, with respect to a patient's arm.

FIG. 4B may depict an embodiment of a UV-permeable-mitten, shown from a top (dorsal) or from a bottom (ventral) view, with respect to a patient's arm.

FIG. 5A may depict an embodiment of a mitten-for-protecting-skin-treatment, shown from a top (dorsal) or from a bottom (ventral) view, with respect to a patient's arm; wherein an outer-elongate-sleeve may be shown in a rolled configuration.

FIG. 5B may depict the embodiment of the mitten-for-protecting-skin-treatment from FIG. 5A, shown from a top (dorsal) or from a bottom (ventral) view, with respect to a patient's arm; but wherein the outer-elongate-sleeve may be shown in a partially unrolled (partially deployed) configuration.

FIG. 5C may depict the embodiment of the mitten-for-protecting-skin-treatment from FIG. 5A, shown from a top (dorsal) or from a bottom (ventral) view, with respect to a patient's arm; but wherein the outer-elongate-sleeve may be shown in a completely unrolled (fully deployed) configuration.

FIG. 6A may depict a longitudinal cross-section through an embodiment of a mitten-with-finger-opening.

FIG. 6B may depict the embodiment of the mitten-with-finger-opening from FIG. 6A, from a ventral (bottom) view.

FIG. 6C may depict the embodiment of the mitten-with-finger-opening from FIG. 6A, from a partial side view and showing an opening-for-fingers.

FIG. 6D may depict the embodiment of the mitten-with-finger-opening from FIG. 6A, shown scrunched up with a thumb passing through an elongate-sleeve and the opening-for-fingers.

FIG. 6E may depict the embodiment of the mitten-with-finger-opening from FIG. 6A, shown scrunched up with fingers passing through the elongate-sleeve and the opening-for-fingers.

FIG. 6F may depict the embodiment of the mitten-with-finger-opening from FIG. 6A, shown scrunched up with fingers passing through the elongate-sleeve and the opening-for-fingers.

FIG. 7A may depict steps in a method of mitigating dissociation of a medical device from a patient.

FIG. 7B may depict steps in a method of mitigating dissociation of a medical device from a patient.

FIG. 7C may depict steps in a method of protecting treated skin of a limb.

FIG. 8 may depict a patient-mitten being properly worn over a majority of a limb (e.g., a left arm) of a given patient.

FIG. 9A may depict a bottom (ventral) view of a mitten-with-finger-opening, in its fully covered configuration.

FIG. 9B may depict a top (dorsal) view of the mitten-with-finger-opening of FIG. 9A, in its fully covered configuration.

FIG. 9C may depict a side view of the mitten-with-finger-opening of FIG. 9A, in its fully covered configuration.

FIG. 10A may depict a bottom (ventral) view of a mitten-with-finger-opening, in its fully covered configuration, and while being worn.

FIG. 10B may depict a top (dorsal) view of the mitten-with-finger-opening of FIG. 10A, in its fully covered configuration, and while being worn.

FIG. 10C may depict a side view of the mitten-with-finger-opening of FIG. 10A, in its fully covered configuration, and while being worn.

FIG. 11A may depict a bottom (ventral) view of a mitten-with-finger-opening, in its partially covered configuration, and while being worn.

FIG. 11B may depict a top (dorsal) view of the mitten-with-finger-opening of FIG. 11A, in its partially covered configuration, and while being worn.

FIG. 11C may depict a side view of the mitten-with-finger-opening of FIG. 11A, in its partially covered configuration, and while being worn.

FIG. 12A may show portions of elongate-sleeve 103 with at least one access-slit 107 that may be shown removably open.

FIG. 12B may show portions of elongate-sleeve 103 with at least one access-slit 107 that may be removably closed (sealed).

FIG. 13A may depict a top (dorsal) view of a mitten-with-finger-opening, in its partially covered configuration, while being worn, showing at least some fingernails of the patient's hand visible.

FIG. 13B may depict a side view of the mitten-with-finger-opening of FIG. 13A, in its fully covered configuration, and while being worn.

### REFERENCE NUMERAL SCHEDULE

100 patient-mitten 100
101 hand-covering 101
103 elongate-sleeve 103
105 wrist-union 105
107 access-slit 107
109 arm-grabber 109
111 finger-tip-end 111
113 wrist-end 113
121 proximal-end 121
123 distal-end 123
125 complimentary-edges 125
127 slit-fastener 127
129 break-fasteners 129
131 exterior 131
133 tubing-anchor 133
135 tubing-hole 135
137 interior-surface 137
200 mitten-with-attachment-anchors 200
201 attachment-anchors 201
300 mitten-with-pocket 300
301 pocket 301
400 UV-permeable-mitten 400
500 mitten-for-protecting-skin-treatment 500
503 outer-elongate-sleeve 503
504 inner-elongate-sleeve 504
509 arm-grabber 509
510 fastener-for-arm-grabber 510
522 flare-terminal-end 522
531 exterior-of-outer-elongate-sleeve 531
532 exterior-of-inner-elongate-sleeve 532
600 mitten-with-finger-opening 600
601 fully-covered-configuration 601
603 uncovered-configuration 603
605 opening-for-fingers 605
607 cover-flap 607
607a palm-support 607a
609 ventral-side 609 (also known as “palm-side 609”)
611 dorsal-side 611 (also known as “back-of-hand side 611”)
700 method of mitigating dissociation of medical device from patient 700
701 step of attaching medical device to limb of patient 701
703 step of passing portion of medical device through mitten 703
705 step of fitting and securing mitten onto limb with medical device 705
707 step of anchoring region of medical device to mitten 707
710 method of mitigating dissociation of medical device from patient 710
711 step of passing portion of medical device through mitten 711
720 method of protecting treated skin of limb 720
721 step of applying medicament to skin of limb 721
723 step of pulling “inner-elongate-sleeve 504” over limb with medicament 723
725 step of deploying “outer-elongate-sleeve 503” over “inner-elongate-sleeve 504” 725
727 step of securing “mitten-for-protecting-skin-treatment 500”
to limb with medicament 727
800 patient-mitten 800
900 medical tubing 900
950 patient 950
951 shoulder 951
952 upper arm 952
953 armpit region 953
955 elbow region 955
1001 flippable-cover 1001
1003 flippable-cover-end 1003
1005 first-side 1005
1007 second-side 1007
1009 length-of-hand-covering 1009
1011 length-of-flippable-cover 1011
1013 elastic-strap 1013
1015 housing-for-elastic 1015
1017 seam 1017
1105 third-side 1105
1107 fourth-side 1107
1207 flap 1207
1225 region 1225
1231 fastener 1231
1241 complimentary-fastener 1241
1300 patient-mitten 1300

### DETAILED DESCRIPTION OF THE INVENTION

Note, the names (terms) assigned to reference numerals shown in the drawings are intended to convey structural and/or functional information of the given component, part, and/or structure.

Note, the terms “patient” and “wearer” may be used interchangeably herein, as denoting a person wearing a given mitten that is described herein.

In the following discussion that addresses a number of embodiments and applications of the present invention, reference is made to the accompanying drawings that form a part thereof, where depictions are made, by way of illustration, of specific embodiments in which the invention may be practiced. It is to be understood that other embodiments may be utilized and changes may be made without departing from the scope of the invention.

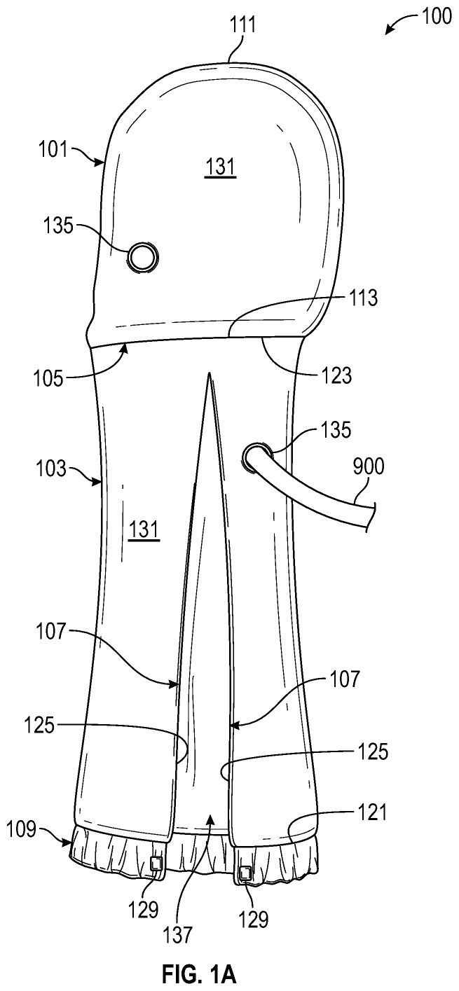

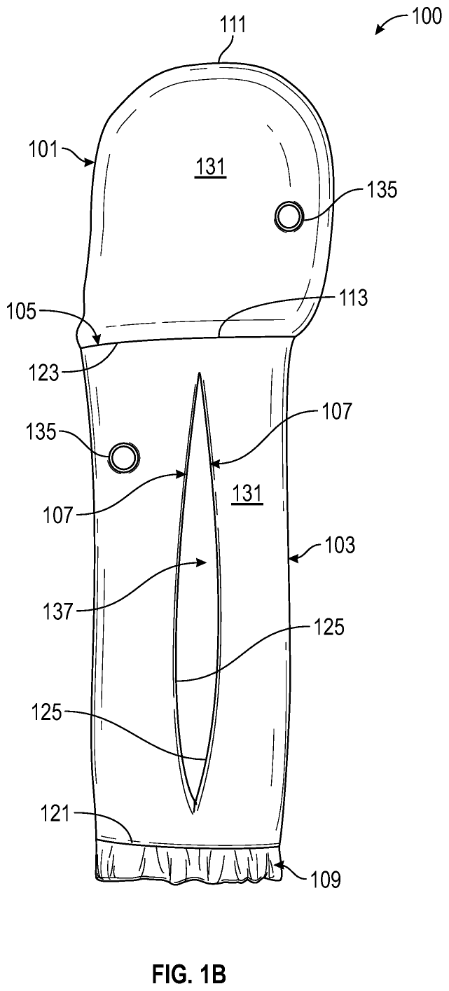

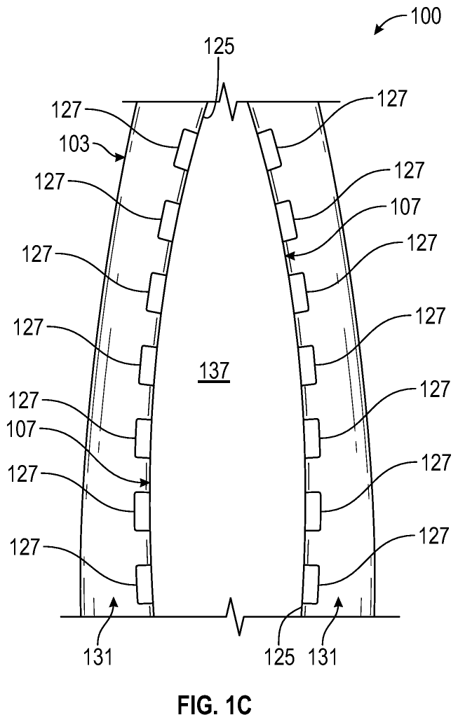

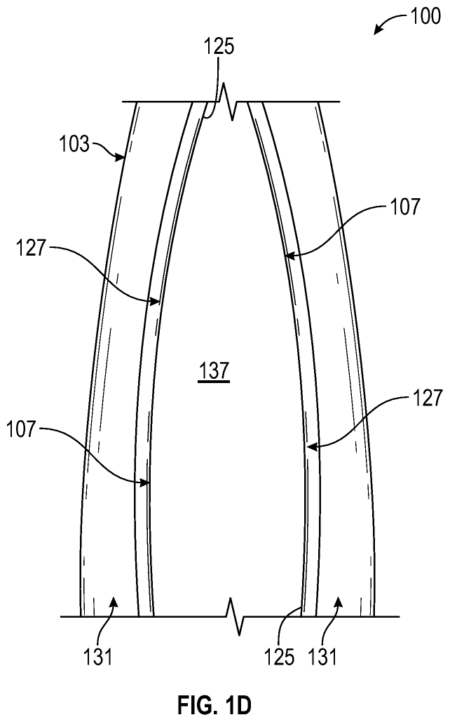

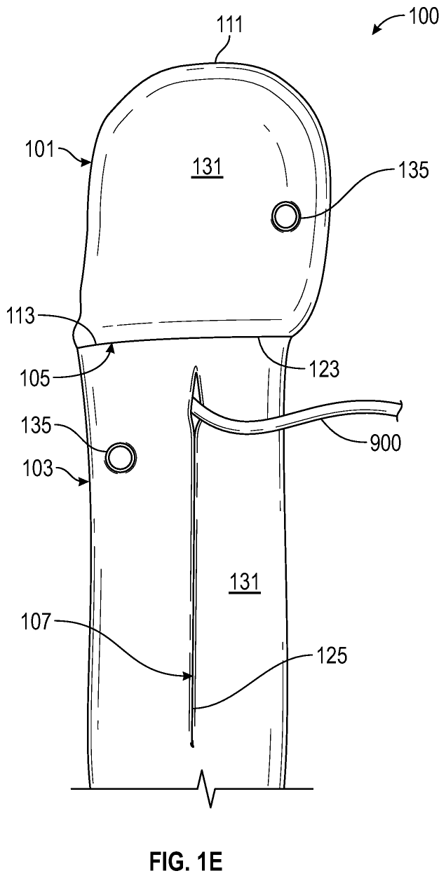

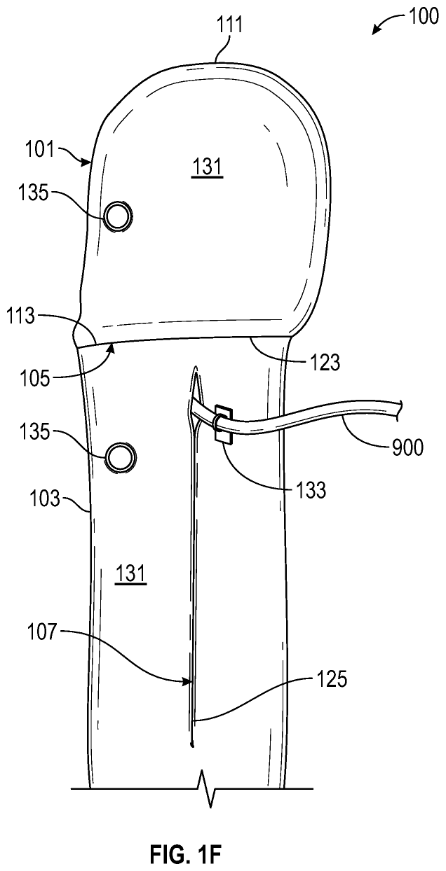

FIG. 1A may depict an embodiment of a patient-mitten 100, shown from a top (dorsal) or from a bottom (ventral) view, with respect to a patient's arm. In some embodiments, patient-mitten 100 may comprise: hand-covering 101, elongate-sleeve 103, wrist-union 105, access-slit 107, and arm-grabber 109. In some embodiments, hand-covering 101 may be sized to substantially cover a hand and fingers of the patient wearing patient-mitten 100. Note, a thumb of a patient's hand may be considered a part of the fingers. In some embodiments, elongate-sleeve 103 may be sized to substantially cover a majority of an arm of the patient. In some embodiments, elongate-sleeve 103 may be sized to cover at least a forearm and an elbow of a given arm of the patient. In some embodiments, hand-covering 101 may be attached to elongate-sleeve 103 at wrist-union 105. In some embodiments, access-slit 107 may be located on elongate-sleeve 103. In some embodiments, access-slit 107 may be for providing access to portions of the arm within elongate-sleeve 103. When access-slit 107 may be removably closed, elongate-member 103 may substantially cover the majority of that arm, such as, at least the forearm and elbow of that arm. Access to otherwise covered portions of the arm may be for placing, removing, securing, monitoring, and/or managing medical devices removably attached to the patient. Such medical devices in this context may be medical tubing 900 (e.g., IV tubing), luer-locks, ports (for connecting to medical tubing 900 or for connecting to syringes), vital sign monitoring devices (e.g., an oxygen sensor, pulse sensor, and the like) and their electrical power cords/cabling/wires, and/or the like. See e.g., FIG. 1A, FIG. 1E and FIG. 1F for a depiction of medical tubing 900. Medical tubing 900 shown in the figures could also be the cabling, wiring, and/or electrical/power/communication cords to various medical devices. In some embodiments, arm-grabber 109 may be for squeezing (gripping) against the arm (e.g., upper arm, above the elbow) when the patient-mitten 100 is properly worn by the patient. Arm-grabber 109 may help to keep patient-mitten 100 properly removably attached to the patient. In some embodiments, arm-grabber 109 may be an elongate member, a hollow annular ring, that may substantially circumscribe a portion of the arm squeezing that portion of the arm. In some embodiments, arm-grabber 109 may be attached to elongate-sleeve 103.

Discussing FIG. 1A, in some embodiments, hand-cover 101 may comprise two opposing ends, a finger-tip-end 111 and a wrist-end 113. When patient-mitten 100 may be being worn properly by the patient, with fingers extended, then finger-tip-end 111 may cover the fingers and be closer to the patient's finger tips than to the patient's wrist of that limb that is wearing the given patient-mitten 100. Similarly, when patient-mitten 100 may be being worn properly by the patient, with fingers extended, then wrist-end 113 may be closer to the patient's wrist than to the patient's finger tips of that limb that is wearing the given patient-mitten 100.

Continuing discussing FIG. 1A, in some embodiments, wrist-union 105 may be located at wrist-end 113. In some embodiments, wrist-union 105 may delineate hand-cover 101 from elongate-sleeve 103. In some embodiments, wrist-union 105 may not be a separate physical structure, but rather may delineate hand-cover 101 from elongate-sleeve 103. In some embodiments, wrist-union 105 may be a physical structure, e.g., a seam, where hand-cover 101 is attached to elongate-sleeve 103. In some embodiments, wrist-union 105 may have elastic properties to at least partially squeeze the wrist of the patient, when the patient-mitten 100 is being properly worn.

Continuing discussing FIG. 1A, in some embodiments, hand-cover 101 may be one or more of: substantially constructed from a fabric, substantially soft, substantially flexible, and/or the like. Hand-cover 101 may function to keep the hand and the fingers substantially covered by hand-cover 101 warm. Hand-cover 101 may function to prevent fingernails of the covered fingers from scratching other parts of the patient or articles or care givers.

In some embodiments, the patient may be a primate. In some embodiments, the patient may be a human. In some embodiments, the patient may be selected from: a baby, a newborn, an infant, a prematurely born infant (i.e., a preemie), a toddler, a child, a kid, a teenager, a youth, an adolescent, a young adult, an adult, and/or the like. In some embodiments, the patient may be a person at risk of scratching themselves with their own fingernails and regardless of their age. In some embodiments, the patient may be a person who has at least one medical device attached to their arm, hand, and/or fingers and regardless of their age. Such medical devices in this context may be medical tubing 900 (e.g., IV tubing), luer-locks, ports (for connecting to medical tubing 900 or for connecting to syringes), vital sign monitoring devices (e.g., an oxygen sensor, pulse sensor, and the like), and/or the like. In some embodiments, the patient may be a person at risk of trauma to their skin and regardless of their age; e.g., those with thin or thinning skin. In some embodiments, the patient may be person wearing a given patient-mitten 100 in order to keep hands and/or fingers warm. And of course, it is expressly contemplated that in some applications of use, a wearer of a given patient-mitten 100 need not be a patient. The mittens and/or patient-mittens disclosed herein are for use on a limb (e.g., including the fingers, hand, wrist, forearm, and elbow of that limb) of a person, e.g., the patient.

Continuing discussing FIG. 1A, in some embodiments, elongate-sleeve 103 may comprise two opposing ends, a proximal-end 121 and a distal-end 123. In some embodiments, arm-grabber 109 may be attached to proximal-end 121 and distal-end 123 may be located at wrist-union 105. In some embodiments, when patient-mitten 100 may be properly worn by the patient, proximal-end 121 may be closest to the bicep/tricep of the patient of the arm that is substantially covered by the elongate-sleeve 103; and distal-end 123 may be closest to wrist-union 105. In some embodiments, when patient-mitten 100 may be properly worn by the patient, proximal-end 121 may be closest to an armpit of the patient of the arm that is substantially covered by the elongate-sleeve 103; and distal-end 123 may be closest to wrist-union 105. In some embodiments, distal-end 123 of elongate-sleeve 103 may be attached to wrist-end 113 of hand-cover 101. In some embodiments, distal-end 123 of elongate-sleeve 103 may be attached to wrist-end 113 of hand-cover 101 at wrist-union 105.

In some embodiments, elongate-sleeve 103 may be one or more of: substantially constructed from a fabric, substantially soft, and/or substantially flexible. In some embodiments, elongate-sleeve 103 may be a hollow elongate member, sized to slip over and cover a majority of the arm of the patient.

Continuing discussing FIG. 1A, in some embodiments, access-slit 107 may comprise a pair of removably sealable complimentary-edges 125. In some embodiments, these complimentary-edges 125 may be removably sealable by a slit-fastener 127 (see e.g., FIG. 1C and FIG. 1C). In some embodiments, slit-fastener 127 may be selected from the group consisting of one or more of: a zipper; a press fit (e.g., a Ziplock type of fastener); a snap fit (e.g., plastic snaps); a button; ties; lacing; a plurality of hooks along one complimentary-edge 125 and a complimentary plurality of loops along the other remaining complimentary-edge 125 (i.e., Velcro or Velcro like); and/or the like.

Continuing discussing FIG. 1A, in some embodiments, elongate-sleeve 103 may comprise one or more of access-slit 107. In some embodiments, access-slit 107 may be located on a dorsal side of elongate-sleeve 103; wherein “dorsal side” is with respect to the patient's arm. In some embodiments, access-slit 107 may be located on a ventral side of the elongate-sleeve 103; wherein “ventral side” is with respect to the patient's arm. In some embodiments, access-slit 107 may run substantially parallel with a length of elongate-sleeve 103. In some embodiments, access-slit 107 may run substantially parallel with a length of the patient's forearm, when that given arm may be removably inserted within elongate-sleeve 103.

Continuing discussing FIG. 1A, in some embodiments, arm-grabber 109 may be an elastic member or substantially an elastic member or a member with elastic properties. In some embodiments, arm-grabber 109 may be a hollow annular ring. In some embodiments, arm-grabber 109 may be at least partially constructed from an elastic band, Spandex, or the like fabrics. In some embodiments, during normal use, arm-grabber 109 may squeeze (grip) around an upper portion of the patient's arm, such as, at or above the elbow. In some embodiments, during normal use, arm-grabber 109 may circumscribe and squeeze (grip) around an upper portion of the patient's arm, such as, at or above the elbow. Use of arm-grabber 109 may be important in keeping a given patient-mitten 100 from slipping down on the arm. Use of arm-grabber 109 may be important in preventing a given patient-mitten 100 from falling off of that limb.

Continuing discussing FIG. 1A, in some embodiments, in some embodiments, hand-cover 101 may comprise one more tubing-holes 135 sized to pass through medical tubing 900. In some embodiments, elongate-sleeve 103 may comprise one more tubing-holes 135 sized to pass through medical tubing 900. In some embodiments, a given patient-mitten 100 may have no such tubing-holes 135; and just use access-slit 107 for tubing passage.

In some embodiments, a given patient-mitten 100 may be constructed such that a ratio of a length of the hand-cover 101 (e.g., wrist-end 113 to finger-tip-end 111) to a length of the elongate-sleeve 103 (e.g., from proximal-end 121 to distal-end 123) may be within a range of 0.32 to 0.34, including the endpoints of that range. In some embodiments, a given patient-mitten 100 may be constructed such that a ratio of a length of the hand-cover 101 to a length of the elongate-sleeve 103 may be within a range of 0.29 to 0.35, including the endpoints of that range. Such rations may provide for arm-grabber 109 being located along the upper arm past the elbow which may be important for minimizing that given patient-mitten 100 from slipping off of that limb.

In some embodiments, hand-cover 101 and/or elongate-sleeve 103 may be substantially ultraviolet (UV) light permeable. In some embodiments, hand-cover 101 and/or elongate-sleeve 103 may be substantially constructed from UV light permeable fabrics. In some embodiments, a majority of exterior 131 may be substantially UV light permeable. In some embodiments exterior 131 may refer to an exterior surface of hand-cover 101 and/or an exterior surface of elongate-sleeve 103. In some embodiments, portions of interior-surface 137 may be substantially UV light permeable. In some embodiments interior-surfaces 137 may refer to an interior surface of hand-cover 101 and/or an interior surface of elongate-sleeve 103. In some embodiments, exterior 131 may be opposing interior-surface 137. In some embodiments, at least some interior-surfaces 137 may be in removable physical skin contact with skin of the fingers, hand, and/or arm that is wearing that given patient-mitten 100. Such UV light permeability may be important for when the patient (e.g., a preemie) is in need of producing their own vitamin D; such as, but not limited to, preemies removably housed within incubators. For example, and without limiting the scope of the present invention, such a UV permeable fabric may be Tan Through or CoolTan or TanMeOn brand of fabric or similar type of fabric. For example, and without limiting the scope of the present invention, such a UV permeable fabric may be a mesh fabric or a fabric with relatively large weave or relatively small thread count.

In some embodiments, it may be elongate-sleeve 103 that may have the substantially ultraviolet (UV) light permeable properties as discussed above and not hand-cover 101. Rather, in such embodiments, hand-cover 101 may be more thick or more plush as compared against elongate-sleeve 103. In some embodiments, hand-cover 101 may be at least thick enough or at least plush enough to minimize the wearer being able to scratch themselves when patient-mitten 100 may be worn; while elongate-sleeve 103 may be comparatively thinner (and/or less plush) with the substantially ultraviolet (UV) light permeable properties. In some embodiments, hand-cover 101 may be at least thick enough or at least plush enough to minimize the wearer being able to grab or interact with objects external to patient-mitten 100; which for example, may be useful in applications with preemies who might otherwise inadvertently grab and remove IV tubing; but wherein elongate-sleeve 103 may be comparatively thinner with the substantially ultraviolet (UV) light permeable properties.

In some embodiments, hand-cover 101 and/or elongate-sleeve 103 may be substantially liquid impermeable (e.g., waterproof or water resistant). In some embodiments, hand-cover 101 and/or elongate-sleeve 103 may be substantially constructed from substantially liquid impermeable fabrics. In some embodiments, a majority of exterior 131 may be substantially liquid impermeable (e.g., waterproof or water resistant); as this may help to keep the arm, hands, and/or fingers dry in wet environments. In some embodiments, portions of interior-surface 137 may be substantially liquid impermeable (e.g., waterproof or water resistant); as this may help to confine skin treatments to the treated skin. For example, and without limiting the scope of the present invention, such a fabric or material of construction for hand-cover 101, elongate-sleeve 103, exterior 131, and/or interior-surface 137 may be: a Gore-Tex fabric or Gore-Tex like fabric; a Conduit fabric from Mountain Hardwear; a NeoShell fabric from Polartec; a AQ2/Aquafoil fabric from Berghaus; an Omni-Dry fabric; and/or the like. Some such fabrics may be air permeable (i.e., air breathable). Some such waterproof breathable fabrics may comprise at least two layers, a first layer often made of nylon or polyester or the like, and sometimes referred to as the “face fabric”; and a second layer, a laminated membrane or coating, usually made of ePTFE (expanded Polytetrafluoroethylene, also known as Teflon) or PU (Polyurethane) or the like.

In some embodiments, hand-cover 101 and/or elongate-sleeve 103 may be substantially air permeable (i.e., air breathable). In some embodiments, hand-cover 101 and/or elongate-sleeve 103 may be substantially constructed from substantially air permeable (i.e., air breathable) fabrics. For example, and without limiting the scope of the present invention, such a fabric may be a Gore-Tex fabric or Gore-Tex like fabric.

In some embodiments, hand-cover 101 and/or elongate-sleeve 103 may be substantially constructed from substantially anti-microbial fabrics. In some embodiments, hand-cover 101 and/or elongate-sleeve 103 may comprise interior-surfaces 137 and/or exteriors 131 that may be substantially anti-microbial. In some embodiments exterior 131 may refer to an exterior surface of hand-cover 101 and/or an exterior surface of elongate-sleeve 103. See e.g., FIG. 1A. In some embodiments, interior-surfaces 137 may refer to an interior surface of hand-cover 101 and/or an interior surface of elongate-sleeve 103. See e.g., FIG. 1A.

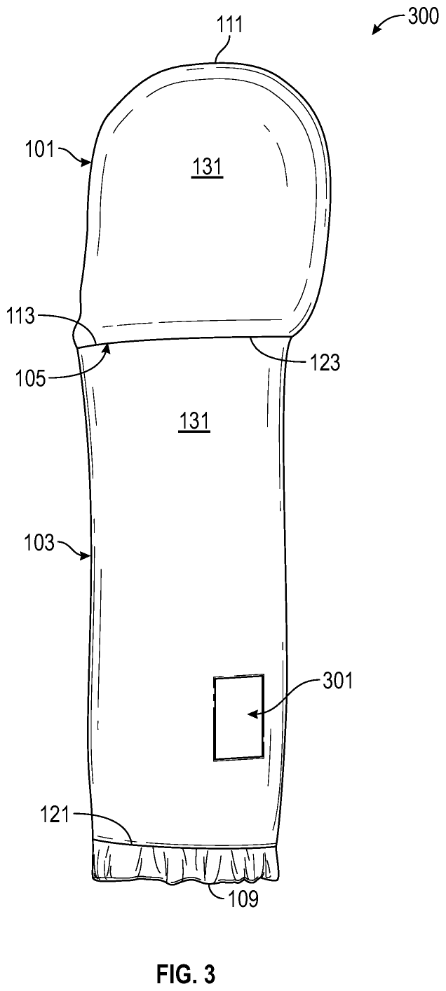

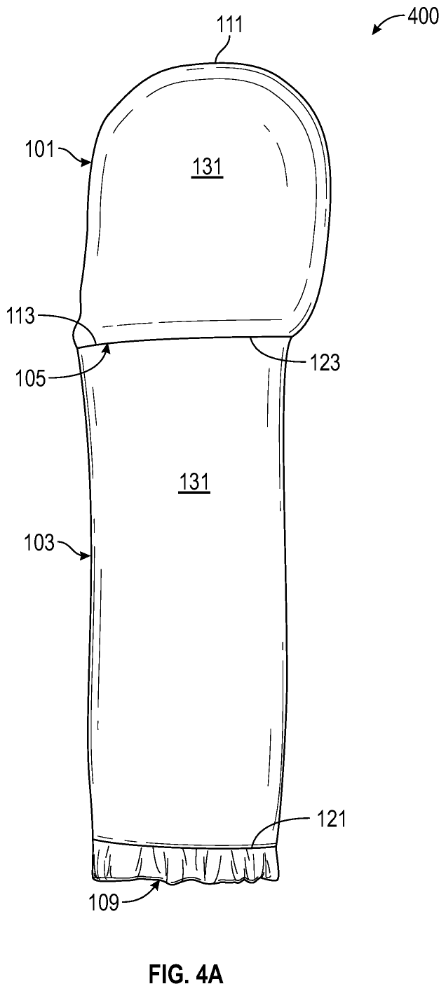

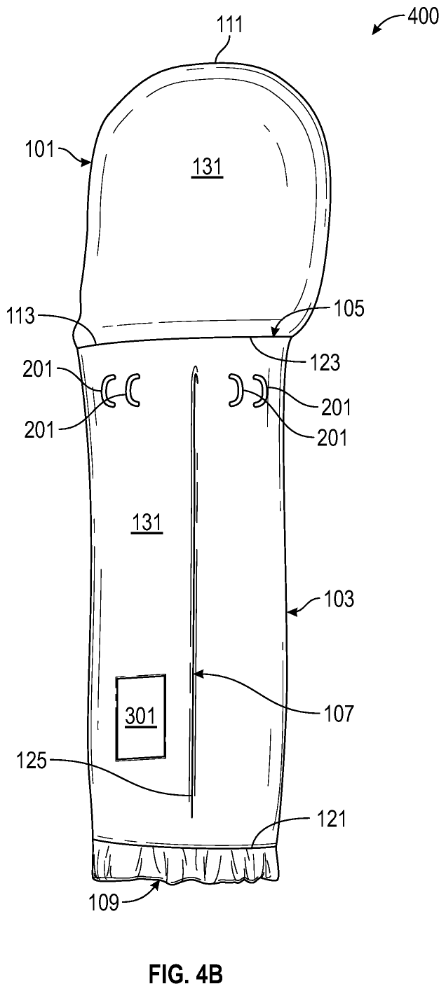

In some embodiments, hand-cover 101 and/or elongate-sleeve 103 comprise one or more pockets 301 located on exterior 131. See e.g., FIG. 3 and FIG. 4B.

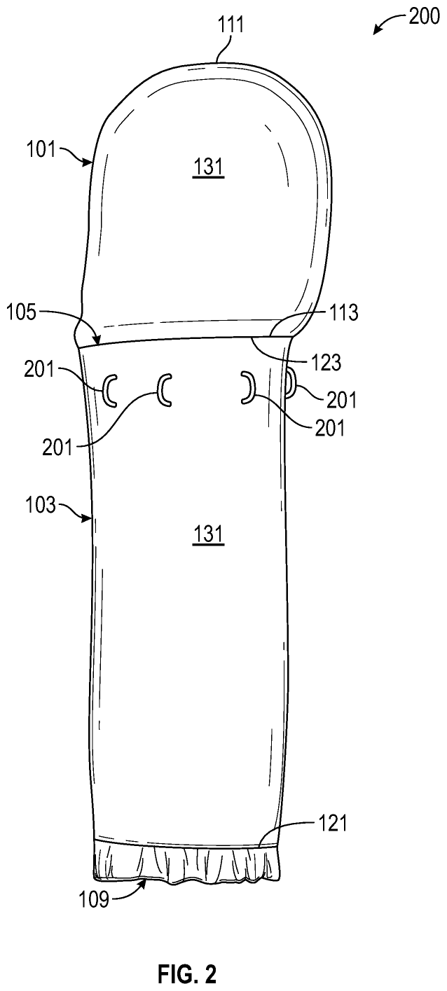

In some embodiments, hand-cover 101 and/or elongate-sleeve 103 may comprise one or more attachment-anchors 201 located on exteriors 131. See e.g., FIG. 2 and FIG. 4B. In some embodiments, attachment-anchors 201 may be partial loop structures or hook structures. In some embodiments, attachment-anchors 201 may provide location(s) on patient-mitten 100 where various articles may be attached to patient-mitten 100. In some embodiments, attachment-anchors 201 may be relatively soft and substantially constructed from an elastomeric material of construction, such as, but not limited to, silicones, rubbers, and/or the like. This may help to avoid injury to the patient. For example, and without limiting the scope of the present invention, such articles that may be attached to various attachment-anchors 201 may be: rings or colorful items for eye and brain stimulation for young children; blinkable lights; teething objects; pacifiers; noise makers; rattles; charms and/or letters for personalization; toys; leashes for attachment to such articles; medical-tubing 900, and/or the like.

Note the above discussion thus far of patient-mitten 100 depicted in FIG. 1A may also apply to patient-mittens 100 embodiments shown in FIG. 1B, FIG. 1C, FIG. 1D, FIG. 1E, and FIG. 1F.

Continuing discussing FIG. 1A, in some embodiments, access-slit 107, when open, may insert an openable break (gap) in arm-grabber 109. In some embodiments, access-slit 107 may run across arm-grabber 109 causing an openable break (gap) in the arm-grabber 109 when access-slot 107 may be open. In some embodiments, the openable break (gap) in arm-grabber 109 may be removably closable by a pair of break-fasteners 129 located at each end of the openable break. In some embodiments, the pair of break-fasteners 129 may be selected from the group consisting of one or more of: a press fit; a snap fit (e.g., plastic snaps); a button; ties; a draw string; a plurality of hooks along at one end of the openable break and a complimentary plurality of loops along the other remaining end of the openable break (i.e., Velcro or Velcro like); and/or the like.

FIG. 1B may depict another embodiment of patient-mitten 100, shown from a top (dorsal) or from a bottom (ventral) view, with respect to a patient's arm. A difference between patient-mitten 100 shown in FIG. 1A versus patient-mitten 100 shown in FIG. 1B, may be in a size and/or location of access-slit 107. For example, and without limiting the scope of the present invention, in FIG. 1A, access-slit 107 may run into and through a portion of arm-grabber 109 causing the openable break (gap) shown between break-fasteners 129 in FIG. 1A; whereas, in FIG. 1B, access-slit 107 may not touch arm-grabber 109.

Discussing FIG. 1B, in some embodiments, access-slit 107 may have a length that is less than a length of elongate-sleeve 103. In some embodiments, access-slit 107 may not run into (i.e., extend into) arm-grabber 109. In some embodiments, access-slit 107 may not touch arm-grabber 109. In some embodiments, access-slit 107 may not interrupt arm-grabber 109.

FIG. 1C may depict an elongate-sleeve 103 portion of patient-mitten 100, depicting part of access-slit 107 and depicting part of slit-fastener 127, shown from a top (dorsal) or from a bottom (ventral) view, with respect to a patient's arm. In some embodiments, slit-fastener 127 may run intermittently at predetermined spacing along each of complimentary-edges 125.

FIG. 1D may depict elongate-sleeve 103 portion of patient-mitten 100, depicting a part of access-slit 107 and depicting a part of another slit-fastener 127, shown from a top (dorsal) or from a bottom (ventral) view, with respect to a patient's arm. In some embodiments, slit-fastener 127 may run substantially along each of complimentary-edges 125.

Recall, in some embodiments, slit-fastener 127 may be selected from the group consisting of one or more of: a zipper; a press fit (e.g., a Ziplock type of fastener); a snap fit (e.g., plastic snaps); a button; ties; lacing; a plurality of hooks along one complimentary-edge 125 and a complimentary plurality of loops along the other remaining complimentary-edge 125 (i.e., Velcro or Velcro like); and/or the like.

FIG. 1E may depict a partial view of patient-mitten 100, shown from a top (dorsal) or from a bottom (ventral) view, with respect to a patient's arm, showing a section of medical tubing 900 emerging from access-slit 107 in elongate-sleeve 103 of that patient-mitten 100. Thus, FIG. 1E may show a function of access-slit 107. As noted, access-slit 107 may provide access to otherwise covered portions of the arm for placing, removing, securing, monitoring, and/or managing medical devices removably attached to the patient's arm, hand, and/or fingers. Such medical devices in this context may be: medical tubing 900 (e.g., IV tubing); luer-locks; ports (for connecting to medical tubing 900 or for connecting to syringes); vital sign monitoring devices (e.g., an oxygen sensor, pulse sensor, and the like) and/or their cords, cables, wires; and/or the like.

FIG. 1F may depict a partial view of patient-mitten 100, shown from a top (dorsal) or from a bottom (ventral) view, with respect to a patient's arm, showing a section of medical tubing 900 emerging from access-slit 107 in elongate-sleeve 103 of that patient-mitten 100. In some embodiments, elongate-sleeve 103 may comprise at least one tubing-anchor 133. In some embodiments, at least one tubing-anchor 133 may be located on exterior 131 of elongate-sleeve 103. In some embodiments, at least one tubing-anchor 133 may be located on exterior 131 of hand-cover 101. In some embodiments, at least one tubing-anchor 133 may be removably attachable to a portion of tubing (e.g., medical tubing 900, such as, but not limited to IV tubing). In some embodiments, when the portion of the tubing may be removably attached to tubing-anchor 133, the tubing may be less likely to become dissociated from patient-mitten 100. That is, use of tubing-anchor 133 to anchor a section of tubing to elongate-sleeve 103, may help to minimize the patient inadvertently pulling the tubing out from its connection to the patient. In some embodiments, structurally, tubing-anchor 133 may be a hook structure and/or a loop structure, sized to removably hold a section of the tubing. In some embodiments, tubing-anchor 133 may be a type of attachment-anchor 201 (see e.g., FIG. 2).

In some embodiments, tubing-anchors 133 may be relatively soft and substantially constructed from an elastomeric material of construction, such as, but not limited to, silicones, rubbers, and/or the like. This may help to avoid injury to the patient.

Patient-mitten 100 partially shown in FIG. 1F may differ from patient-mitten 100 partially shown in FIG. 1E, in that in FIG. 1F, patient-mitten 100 may comprise one or more tubing-anchors 133; whereas, the patient-mitten 100 shown in FIG. 1E may not include any tubing-anchors 133.

In some embodiments, tubing-anchors 133 may be sized to removably capture a section of wires, cables, and/or cords from a medical device attached to the patient's arm, hand, and/or fingers, such as wires, cables, and/or cords from a pulse monitoring sensor or wires, cables, and/or cords from an oxygen level monitoring sensor.

FIG. 2 may depict an embodiment of a mitten-with-attachment-anchors 200, shown from a top (dorsal) or from a bottom (ventral) view, with respect to a patient's arm, wherein at least one attachment-anchor 201 may be shown attached to elongate-sleeve 103 of mitten-with-attachment-anchors 200. In some embodiments, mitten-with-attachment-anchors 200 may comprise: hand-covering 101, elongate-sleeve 103, wrist-union 105, and arm-grabber 109 as these structures were discussed above. Some embodiments of mitten-with-attachment-anchors 200 may not include access-slit 107. Some embodiments of mitten-with-attachment-anchors 200 may also comprise access-slit 107 located on elongate-sleeve 103.

Discussing FIG. 2, in some embodiments, hand-cover 101 and/or elongate-sleeve 103 may comprise one or more attachment-anchors 201 located on exteriors 131. See also, FIG. 4B. In some embodiments, attachment-anchors 201 may be partial loop structures or hook structures. In some embodiments, attachment-anchors 201 may provide a location(s) on mitten-with-attachment-anchors 200 where various articles may be attached to mitten-with-attachment-anchors 200. In some embodiments, attachment-anchors 201 may be relatively soft and substantially constructed from an elastomeric material of construction, such as, but not limited to, silicones, rubbers, and/or the like. This may help to avoid injury to the patient.

FIG. 3 may depict an embodiment of a mitten-with-pocket 300, shown from a top (dorsal) or from a bottom (ventral) view, with respect to a patient's arm, wherein at least one pocket 301 may be shown attached to elongate-sleeve 103 of mitten-with-pocket 300. In some embodiments, hand-cover 101 and/or elongate-sleeve 103 may comprise one or more pockets 301 located on exteriors 131. See also, FIG. 4B. Such pockets 301 may be for removable storage of various articles. In some embodiments, pocket 301 may be one or more of: substantially constructed from a fabric, substantially soft, and/or substantially flexible.

In some embodiments, mitten-with-pocket 300 may comprise: hand-covering 101, elongate-sleeve 103, wrist-union 105, and arm-grabber 109 as these structures were discussed above. Some embodiments of mitten-with-pocket 300 may not include access-slit 107. Some embodiments of mitten-with-pocket 300 may also comprise access-slit 107 located on elongate-sleeve 103.

FIG. 4A may depict an embodiment of a UV-permeable-mitten 400, shown from a top (dorsal) or from a bottom (ventral) view, with respect to a patient's arm. In some embodiments, UV-permeable-mitten 400 may comprise: hand-covering 101, elongate-sleeve 103, wrist-union 105, and arm-grabber 109 as these structures were discussed above. Some embodiments of UV-permeable-mitten 400 may not include access-slit 107. Some embodiments of UV-permeable-mitten 400 may also comprise access-slit 107 located on elongate-sleeve 103.

Discussing FIG. 4A, in some embodiments, hand-cover 101 and/or elongate-sleeve 103 may be substantially ultraviolet (UV) light permeable. In some embodiments, hand-cover 101 and/or elongate-sleeve 103 may be substantially constructed from ultraviolet (UV) light permeable fabrics. This may be important for when the patient is in need of producing their own vitamin D; such as, but not limited to, preemies removably housed within incubators. For example, and without limiting the scope of the present invention, such a fabric may be a mesh fabric or a fabric with relatively large weave or relatively small thread count.

FIG. 4B may depict another embodiment of a UV-permeable-mitten 400, shown from a top (dorsal) or from a bottom (ventral) view, with respect to a patient's arm. In some embodiments, UV-permeable-mitten 400 may comprise: hand-covering 101, elongate-sleeve 103, wrist-union 105, and arm-grabber 109. In some embodiments, UV-permeable-mitten 400 may also comprise one or more of: access-slit 107, tubing-hole 135, attachment-anchor 201, pocket 301 as these structures were discussed above.

Discussing FIG. 4B, in some embodiments, hand-cover 101 and/or elongate-sleeve 103 may comprise one or more attachment-anchors 201 located on exteriors 131. See also, FIG. 2. In some embodiments, attachment-anchors 201 may be partial loop structures or hook structures. In some embodiments, attachment-anchors 201 may provide location(s) on UV-permeable-mitten 400 where various articles may be attached to UV-permeable-mitten 400. In some embodiments, attachment-anchors 201 may be relatively soft and substantially constructed from an elastomeric material of construction, such as, but not limited to, silicones, rubbers, and/or the like. This may help to avoid injury to the patient.

Continuing discussing FIG. 4B, in some embodiments, hand-cover 101 and/or elongate-sleeve 103 may comprise one or more pockets 301 located on exteriors 131. See also, FIG. 3.

Continuing discussing FIG. 4B, in some embodiments, hand-cover 101 and/or elongate-sleeve 103 may be substantially ultraviolet (UV) light permeable. In some embodiments, hand-cover 101 and/or elongate-sleeve 103 may be substantially constructed from ultraviolet (UV) light permeable fabrics. This may be important for when the patient is in need of producing their own vitamin D; such as, but not limited to, preemies removably housed within incubators. For example, and without limiting the scope of the present invention, such a fabric may be a mesh fabric or a fabric with relatively large weave or relatively small thread count.

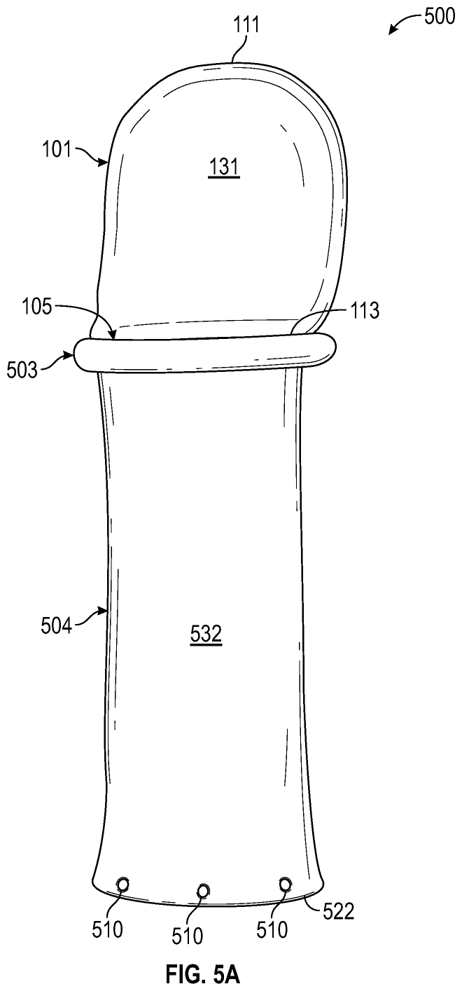

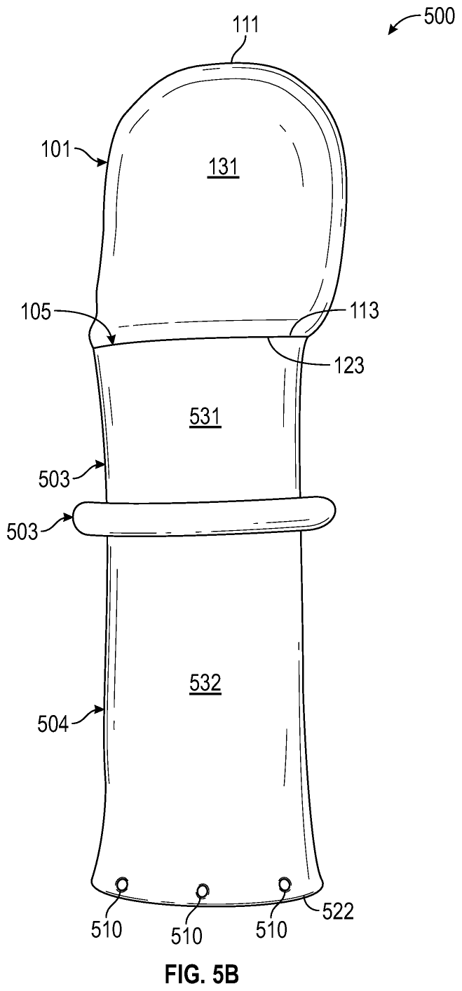

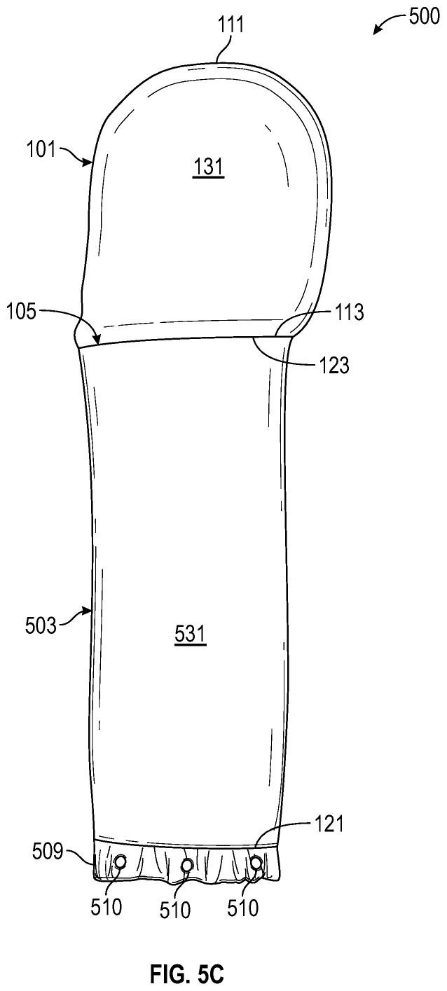

FIG. 5A may depict an embodiment of a mitten-for-protecting-skin-treatment 500, shown from a top (dorsal) or from a bottom (ventral) view, with respect to a patient's arm; wherein an outer-elongate-sleeve 503 may be shown in a rolled configuration. FIG. 5B may depict mitten-for-protecting-skin-treatment 500, shown from a top (dorsal) or from a bottom (ventral) view, with respect to a patient's arm; but wherein outer-elongate-sleeve 503 may be shown in a partially unrolled (partially deployed) configuration. FIG. 5C may depict mitten-for-protecting-skin-treatment 500, shown from a top (dorsal) or from a bottom (ventral) view, with respect to a patient's arm; but wherein outer-elongate-sleeve 503 may be shown in a completely unrolled (fully deployed) configuration. Thus, FIG. 5B may depict a transitional configuration between the configurations depicted in FIG. 5A and as depicted in FIG. 5C.

In some embodiments, mitten-for-protecting-skin-treatment 500 may comprise: hand-covering 101, outer-elongate-sleeve 503, inner-elongate-sleeve 504, wrist-union 105, and arm-grabber 509. See e.g., FIG. 5A, FIG. 5B, and/or FIG. 5C. Hand-covering 101, wrist-union 105, finger-tip-end 111, wrist-end 113, proximal-end 121, distal-end 123, and exterior 131 shown in FIG. 5A, FIG. 5B, or FIG. 5C may be structures and/or regions as discussed above, except as noted here in this discussion of FIG. 5A, FIG. 5B, and FIG. 5C. In some embodiments, hand-covering 101 may be attached to outer-elongate-sleeve 503 and to inner-elongate-sleeve 504 at wrist-union 105. In some embodiments, wrist-union 105 may be located at wrist-end 113. In some embodiments, wrist-union 105 may delineate hand-cover 101 from outer-elongate-sleeve 503 and/or from inner-elongate-sleeve 504. In some embodiments, wrist-union 105 may not be a separate physical structure, but rather may delineate hand-cover 101 from outer-elongate-sleeve 503 and/or from inner-elongate-sleeve 504. In some embodiments, wrist-union 105 may be a physical structure, e.g., a seam, where hand-cover 101 is attached to outer-elongate-sleeve 503 and attached to inner-elongate-sleeve 504. In some embodiments, proximal-end 121 and opposing distal-end 123 may be with respect to outer-elongate-sleeve 503. In some embodiments, outer-elongate-sleeve 503 may comprise two opposing ends, proximal-end 121 and distal-end 123. In some embodiments, arm-grabber 509 may be attached to proximal-end 121 and distal-end 123 may be located at wrist-union 105. In FIG. 5A, FIG. 5B, and FIG. 5C exterior 131 may be limited to the exterior of hand-covering 101.

In some embodiments of mitten-for-protecting-skin-treatment 500, elongate-sleeve 103 may be replaced with two layers of elongate sleeves, that of outer-elongate-sleeve 503 and of inner-elongate-sleeve 504. Or alternatively, in some embodiments, elongate-sleeve 103 may be two layers, that of outer-elongate-sleeve 503 and of inner-elongate-sleeve 504. See e.g., FIG. 5A, FIG. 5B, and FIG. 5C. In some embodiments, together outer-elongate-sleeve 503 and of inner-elongate-sleeve 504 may serve a same purpose or a same function as that of elongate-sleeve 103; such as, to cover a majority of the arm wearing the given mitten-for-protecting-skin-treatment 500. Based upon a shared longitudinal axis, outer-elongate-sleeve 503 and of inner-elongate-sleeve 504 may be substantially concentric with respect to each other. When mitten-for-protecting-skin-treatment 500 may be being worn by the patient and outer-elongate-sleeve 503 may be completely deployed (unrolled), interior portions of inner-elongate-sleeve 504 (not shown) may be in physical contact with arm skin of the patient, with exterior-of-outer-elongate-sleeve 531 externally visible.

As noted by the name of “mitten-for-protecting-skin-treatment 500,” mitten-for-protecting-skin-treatment 500 may be a type of patient mitten specifically for protecting the treated skin of the arm, hand, and/or fingers. Mitten-for-protecting-skin-treatment 500 may accomplish this function by interior surfaces of inner-elongate-sleeve 504 that may be in physical contact with skin of the arm, being designed not to absorb nor wick away creams, lotions, salves, ointments, and/or medicaments that may have been applied to the skin of the fingers, hand, and/or arm. Additionally, inner-elongate-sleeve 504 may protect such treated skin from coming into physical contact with environmental articles, which might otherwise remove portions of such creams, lotions, salves, ointments, and/or medicaments from the treated skin (e.g., from brushing against such environmental articles). Additionally, inner-elongate-sleeve 504 may create a controlled, protected, and contained region around the treated skin, which for example, may minimize treated skin from drying out or the applied creams, lotions, salves, ointments, and/or medicaments from evaporating. In some embodiments, the medicament may be selected from one or more of: a topical medication, a cream, a lotion, a salve, an ointment, a gel, a foam, a film, an oil, an essential oil, an unguent, a balm, or an emulsion, portions thereof, combinations thereof, and/or the like.

In some embodiments, inner-elongate-sleeve 504 may be substantially liquid impermeable (e.g., waterproof or water resistant). In some embodiments, inner-elongate-sleeve 504 may be substantially constructed from substantially liquid impermeable fabrics. In some embodiments, a majority of interior surface of inner-elongate-sleeve 504 may be substantially liquid impermeable (e.g., waterproof or water resistant); as this may help to confine skin treatments to the treated skin; and/or as this may help to keep the arm, hands, and/or fingers dry in wet environments. For example, and without limiting the scope of the present invention, such a fabric or material of construction for inner-elongate-sleeve 504 may be: a Gore-Tex fabric or Gore-Tex like fabric; a Conduit fabric from Mountain Hardwear; a NeoShell fabric from Polartec; a AQ2/Aquafoil fabric from Berghaus; an Omni-Dry fabric; and/or the like. Some such fabrics may be air permeable (i.e., air breathable); but, substantially liquid impermeable (e.g., waterproof or water resistant). Some such waterproof breathable fabrics (e.g., inner-elongate-sleeve 504) may comprise at least two layers, a first layer often made of nylon or polyester or the like, and sometimes referred to as the “face fabric”; and a second layer, a laminated membrane or coating, usually made of ePTFE (expanded Polytetrafluoroethylene, also known as Teflon) or PU (Polyurethane) or the like.

In some embodiments, inner-elongate-sleeve 504 may be substantially air permeable (i.e., air breathable). In some embodiments, inner-elongate-sleeve 504 may be substantially constructed from substantially air permeable (i.e., air breathable) fabrics. For example, and without limiting the scope of the present invention, such a fabric may be a Gore-Tex fabric or Gore-Tex like fabric.

In some embodiments, inner-elongate-sleeve 504 may be substantially constructed from substantially anti-microbial fabrics and/or from fabrics treated with anti-microbial properties.

In some embodiments, due to substantially liquid impermeable qualities of inner-elongate-sleeve 504, inner-elongate-sleeve 504 may be more rigid (e.g., less flexible and/or more stiff) than outer-elongate-sleeve 503. Because of this, in some embodiments, one terminal end of inner-elongate-sleeve 504 may flare out (i.e., may be wider), to provide for easier fitting of inner-elongate-sleeve 504 over the patient's arm. See e.g., FIG. 5A and FIG. 5B. That is, at flare-terminal-end 522, a diameter of inner-elongate-sleeve 504 may be greater than a diameter of inner-elongate-sleeve 504 at other locations of inner-elongate-sleeve 504. In some embodiments, flare-terminal-end 522 may be a terminal end of inner-elongate-sleeve 504 that is closest to an armpit (or shoulder or bicep/tricep) of the patient when mitten-for-protecting-skin-treatment 500 may be properly worn on that given arm of that armpit. In some embodiments, flare-terminal-end 522 may be disposed opposite from wrist-union 105. See e.g., FIG. 5A and FIG. 5B.

In some embodiments, inner-elongate-sleeve 504 may flare at flare-terminal-end 522 to fit over (e.g., accommodate) various wraps and/or dressings that might have been applied to the patient's skin of the limb. For example, and without limiting the scope of the present invention, treating dry skin regions and/or eczema regions, a wet and/or mediated wrap or dressing may be applied to the dry skin region and/or eczema region. And the flare at flare-terminal-end 522 of inner-elongate-sleeve 504 may help inner-elongate-sleeve 504 fit over such wraps and/or dressings. See e.g., FIG. 5A and FIG. 5B.

In some embodiments, flare-terminal-end 522 may comprise fastener-for-arm-grabber 510. In some embodiments, arm-grabber 509 (shown in FIG. 5C) may comprise fastener-for-arm-grabber 510. In some embodiments, the fastener-for-arm-grabber 510 of arm-grabber 509 may removably attach to the fastener-for-arm-grabber 510 of flare-terminal-end 522; that is, these respective fastener-for-arm-grabber 510 may be complimentary to each other. See e.g., FIG. 5A, FIG. 5B, and FIG. 5C.

In some embodiments, fastener-for-arm-grabber 510 may be selected from the group consisting of one or more of: a press fit; a snap fit (e.g., plastic snaps); a button; ties; a draw string; a plurality of hooks along at one end of the openable break and a complimentary plurality of loops along the other remaining end of the openable break (i.e., Velcro or Velcro like); and/or the like.

In some embodiments, in operation, once inner-elongate-sleeve 504 may be deployed substantially over the given arm (and potentially over treated skin), then outer-elongate-sleeve 503 may be unrolled from the wrist region to towards the armpit region over both portions of that arm as well as over portions of inner-elongate-sleeve 504; with portions of inner-elongate-sleeve 504 disposed between outer-elongate-sleeve 503 and the skin of that now covered arm; and then the fastener-for-arm-grabber 510 of arm-grabber 509 may removably attach to the fastener-for-arm-grabber 510 of flare-terminal-end 522, past the elbow (proximate to the bicep/tricep). In some embodiments, outer-elongate-sleeve 503 may be softer and/more flexible as compared against inner-elongate-sleeve 504. This dual layer configuration (e.g., of outer-elongate-sleeve 503 and of inner-elongate-sleeve 504) may be desirable because inner-elongate-sleeve 504 may be more stiff and/or more rigid as compared against outer-elongate-sleeve 503.

In some embodiments, arm-grabber 509 may function substantially similar to embodiments of arm-grabber 109. In some embodiments, arm-grabber 509 may comprise substantially similar structures to embodiments of arm-grabber 109. In some embodiments, arm-grabber 509 may be for squeezing (gripping) against the arm when mitten-for-protecting-skin-treatment 500 is properly worn by the patient. Arm-grabber 509 may help to keep mitten-for-protecting-skin-treatment 500 properly removably attached to the patient. In some embodiments, arm-grabber 509 may be an elongate member, a hollow annular ring, that may substantially circumscribe a portion of the arm squeezing that portion of the arm. In some embodiments, arm-grabber 509 may be attached to outer-elongate-sleeve 503. See e.g., FIG. 5C. In some embodiments, arm-grabber 509 may be attached to proximal-end 121 and distal-end 123 may be located at wrist-union 105. In some embodiments, arm-grabber 509 may be an elastic member or substantially an elastic member or a member with elastic properties. In some embodiments, arm-grabber 509 may be a hollow annular ring. In some embodiments, arm-grabber 509 may be at least partially constructed from an elastic band, Spandex, or the like fabrics. Use of arm-grabber 509 may be important in keeping a given mitten-for-protecting-skin-treatment 500 from slipping down on the arm. Use of arm-grabber 509 may be important in preventing a given mitten-for-protecting-skin-treatment 500 from falling off of that limb. See e.g., FIG. 5C.

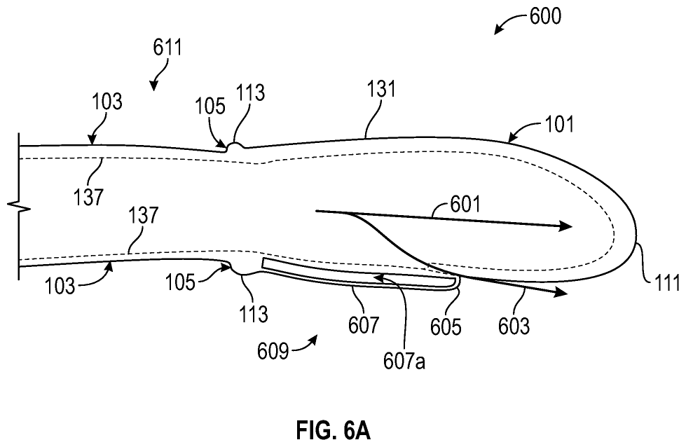

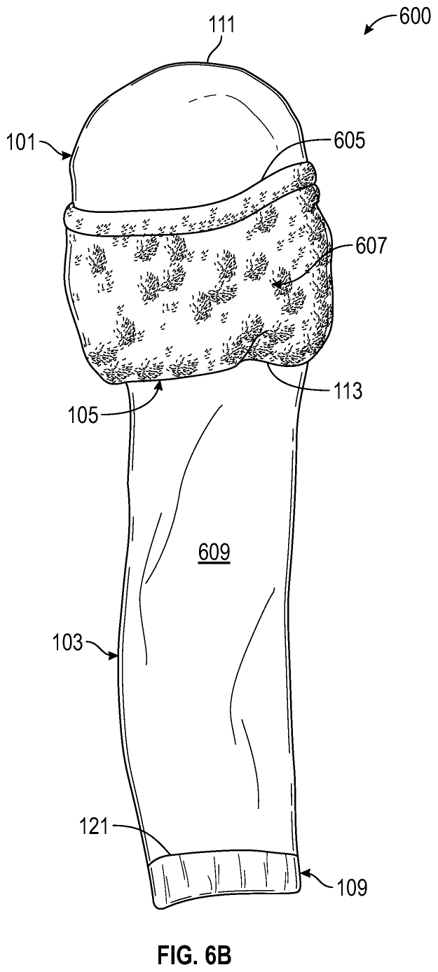

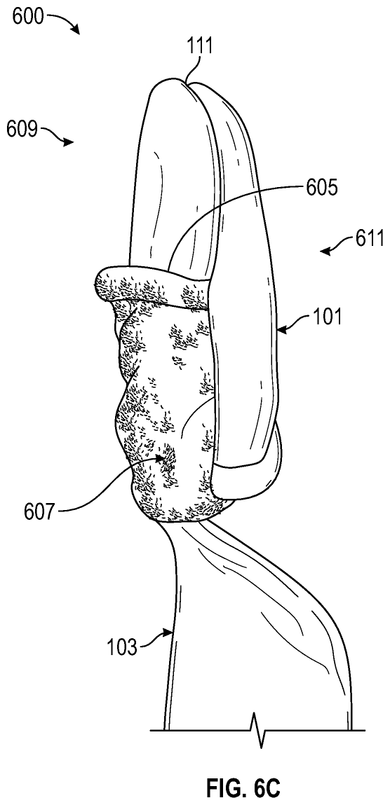

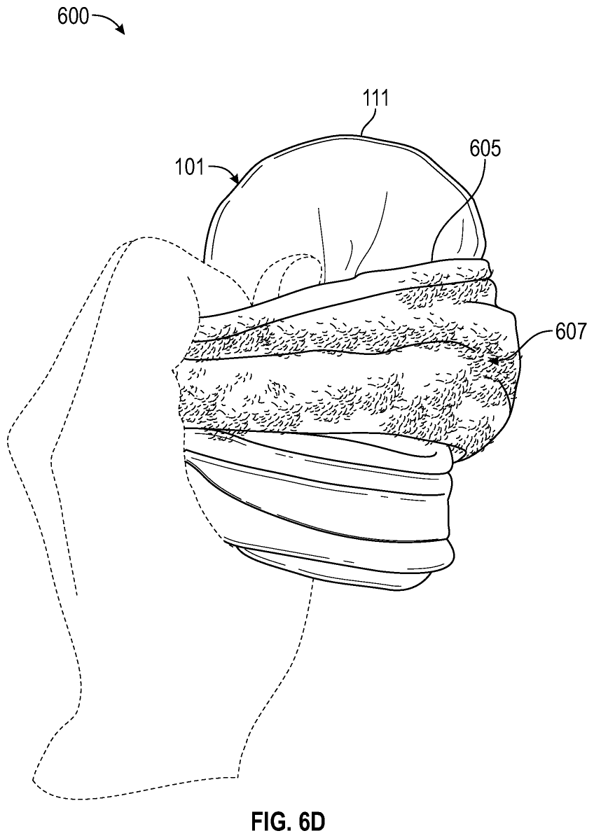

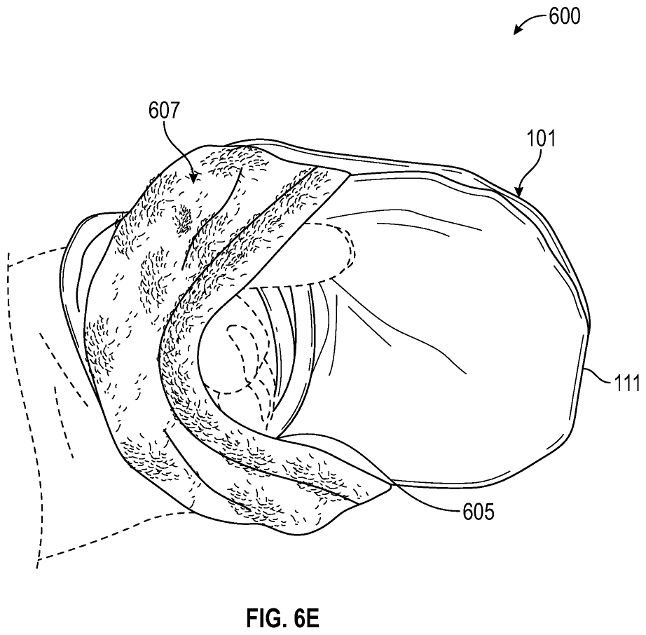

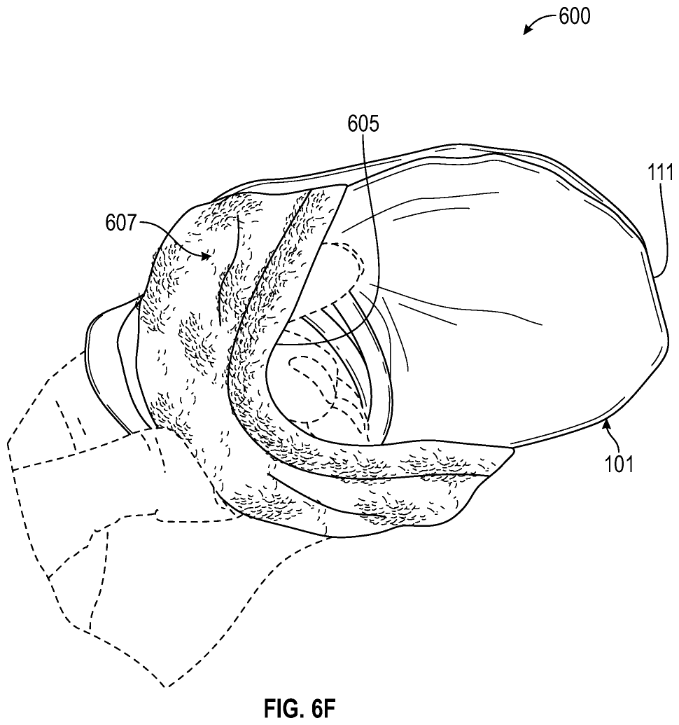

FIG. 6A through FIG. 6F may depict a mitten-with-finger-opening 600; wherein fingers of the wearer (the patient) may be entirely covered by mitten-with-finger-opening 600 or wherein at least some portions of those fingers may be uncovered, but while mitten-with-finger-opening 600 remains otherwise worn. FIG. 6A may depict a longitudinal cross-section through an embodiment of mitten-with-finger-opening 600. FIG. 6B may depict the embodiment of mitten-with-finger-opening 600, from a ventral (bottom) view, i.e., ventral-side 609. FIG. 6C may depict the embodiment of mitten-with-finger-opening 600, from a partial side view and showing an opening-for-fingers 605. FIG. 6D may depict the embodiment of mitten-with-finger-opening 600, shown scrunched up with a thumb passing through an elongate-sleeve 103 and the opening-for-fingers 605. FIG. 6E may depict the embodiment of mitten-with-finger-opening 600, shown scrunched up with fingers passing through the elongate-sleeve 103 and the opening-for-fingers 605. FIG. 6F may depict the embodiment of mitten-with-finger-opening 600, shown scrunched up with fingers passing through the elongate-sleeve 103 and the opening-for-fingers 605.

As shown in FIG. 6A, the fingers of the wearer (the patient) may be entirely covered by mitten-with-finger-opening 600, wherein this is represented by an arrow 601 in FIG. 6A. Arrow 601 represents fully-covered-configuration 601; i.e., wherein the fingers may be entirely covered by hand-cover 101. Similarly, in FIG. 6A, arrow 603 represents uncovered-configuration 603 where at least portions of the wearer's fingers may extend beyond opening-for-fingers 605. Regardless if the finger-tips are following the configuration of arrow 601 (completely covered fingers) or that of arrow 603 (uncovered finger-tips), the palm region of the hand may be still be retained within hand-cover 101.

Continuing discussing FIG. 6A, in some embodiments, disposed between interior-surface 137 and cover-flap 607 may be a palm-support 607a. In some embodiments, palm-support 607a may be configured to provide some rigid support to the wearer's palm (and/or to the wearer's fingers). In some embodiments, palm-support 607a may prevent or minimize the wearer's palm/hand/fingers from curling in on itself (which may be beneficial to those with contracture or the like). In some embodiments, palm-support 607a may be thicker than other regions/portions of the given mitten (e.g., mitten-with-finger-opening 600) to accomplish its supportive role. In some embodiments, palm-support 607a may be more rigid/firmer than other regions/portions of the given mitten (e.g., mitten-with-finger-opening 600) to accomplish its supportive role. In some embodiments, palm-support 607a may be constructed from one or more rigid to semi-rigid materials.

In some embodiments, mitten-with-finger-opening 600 may comprise: hand-covering 101, elongate-sleeve 103, wrist-union 105, and arm-grabber 109. See e.g., FIG. 6A and FIG. 6B. Hand-covering 101, elongate-sleeve 103, wrist-union 105, arm-grabber 109, finger-tip-end 111, wrist-end 113, proximal-end 121, distal-end 123, exterior 131, interior-surface 137 shown in FIG. 6A or FIG. 6B may be structures and/or regions as discussed above, except as noted here in this discussion of FIG. 6A through FIG. 6F. In some embodiments, mitten-with-finger-opening 600 may comprise dorsal-side 611 and opposing to dorsal-side 611 a ventral-side 609. When mitten-with-finger-opening 600 may be properly worn, dorsal-side 611 may be closer to a top (dorsal side) of the wearer's arm that is wearing mitten-with-finger-opening 600; whereas, ventral-side 609 may be closer to a bottom (ventral side) of the wearer's arm that is wearing mitten-with-finger-opening 600. In some embodiments, hand-covering 101 may comprise opening-for-fingers 605 and cover-flap 607. In some embodiments, cover-flap 607 and opening-for-fingers 605 may be located on ventral-side 609 of hand-covering 101. In some embodiments, cover-flap 607 may be a flap of material (e.g., a flap of fabric material) that covers a portion of ventral-side 609 of hand-covering 101. In some embodiments, cover-flap 607 may cover what otherwise would be a hole to interior-surface 137 of hand-covering 101. In some embodiments, at one end of cover-flap 607, closest to finger-tip-end 111, may be opening-for-fingers 605. In some embodiments, around an outside perimeter of cover-flap 607, cover-flap 607 may be attached to exterior 131 of dorsal-side 609 of hand-covering 101, except along opening-for-fingers 605. In some embodiments, opening-for-fingers 605 may be an opening to interior-surface 137 of hand-covering 101. Using these structures, the wearer (e.g., the patient) may have their fingers totally (completely covered) as indicated by fully-covered-configuration 601 or at least partially uncovered, as indicated by uncovered-configuration 603. See e.g., FIG. 6A through FIG. 6F.

Note, it is expressly contemplated and included that embodiments of the present invention may comprise features and/or structures of the various mittens disclosed in a combination fashion. That is, the structures and/or features of mittens 100, 200, 300, 400, 500, and/or 600 may be mixed and/or combined. That is, in some embodiments, any of the disclosed mittens (e.g., 100, 200, 300, 400, 500, and/or 600) may comprise one or more of: access-slit 107; tubing-hole 135; tubing-anchor 133; attachment-anchor 201; pocket 301; outer-elongate-sleeve 503 and inner-elongate-sleeve 504; and/or hand-covering 101 with opening-for-fingers 605 and cover-flap 607.

Also note, while mittens 100, 200, 300, 400, 500, and/or 600 may be used for patients in medical settings and/or environments, it is also expressly contemplated that such mittens may be used by non-patient wearers of such mittens. It is also expressly contemplated that such mittens may be worn by wearers of any age.

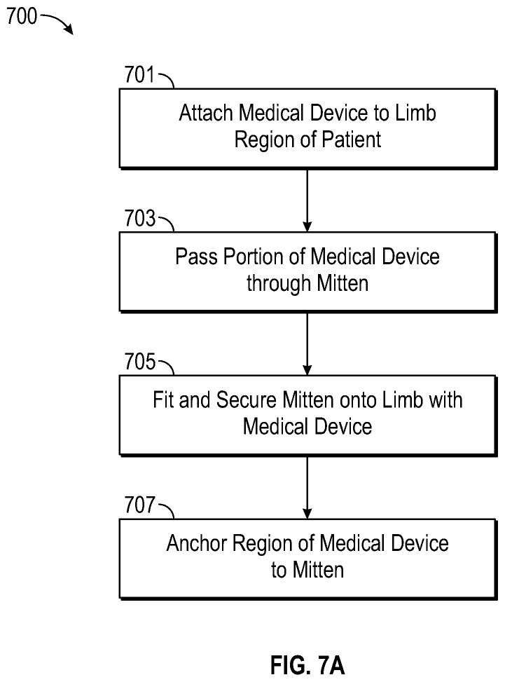

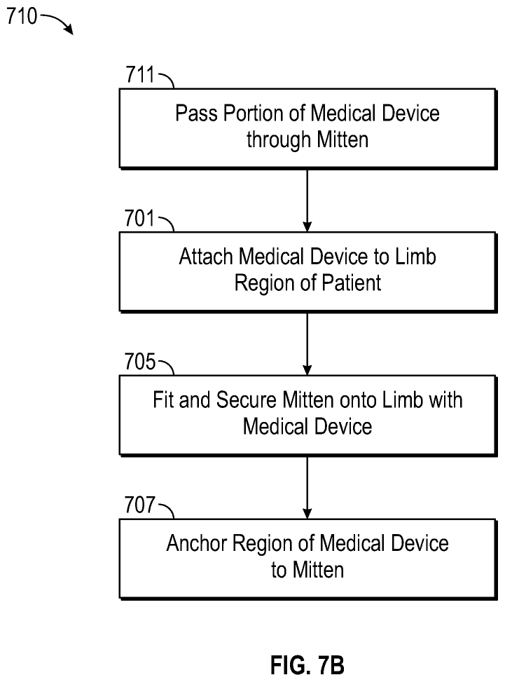

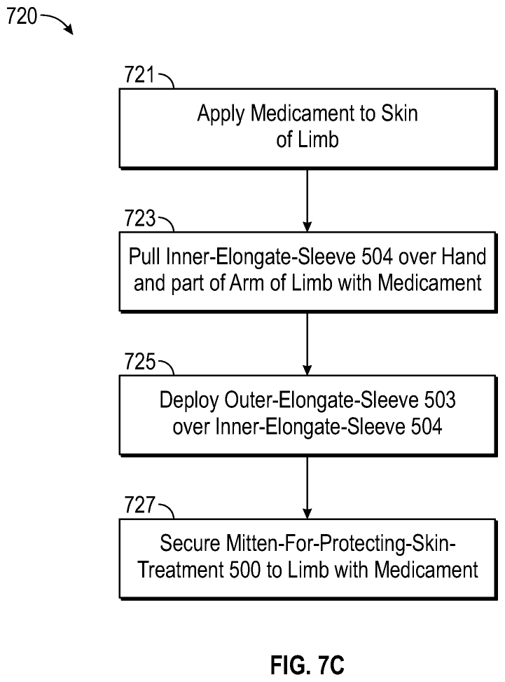

FIG. 7A may depict steps in a method of mitigating dissociation of a medical device from a patient. The method shown in FIG. 7A may be method 700. In some embodiments, method 700 may comprise the steps of: step 701, step 703, step 705, and step 707. In some embodiments, step 701 may be a step of attaching a medical device to a limb region (e.g., finger(s), hand, and/or arm) of a patient. Such medical devices may be as noted above. In some embodiments, step 703 may be a step of passing a portion (e.g., medical-tubing 900, wiring, cabling, cords, etc.) of the medical device through a mitten. In some embodiments, the region of the mitten that is receiving the portion of the medical device may be the access-slit 107, e.g., as shown in FIGS. 1E and 1n FIG. 1F. In some embodiments, step 705 may be a step of fitting and securing the mitten onto the limb with the medical device; such as, by pulling hand-cover 101 over the hand, elongate-sleeve 103 over the forearm and elbow, and having arm-grabber 109 squeeze the upper arm. In some embodiments, step 707 may be a step of anchoring a region of the medical device to the mitten. The anchoring noted in step 707 may be via tubing-anchor 133 shown in FIG. 1F; attachment-anchors 201 shown in FIG. 2 and FIG. 4B; and/or via tape, rubber band, an elastic strip, string, rope, cordage, and the like.

In some embodiments, the mitten noted in method 700 may be patient-mitten 100, mitten-with-attachment-anchors 200, mitten-with-pocket 300, UV-permeable-mitten 400, mitten-for-protecting-skin-treatment 500, mitten-with-finger-opening 600, mitten 800 or patient-mitten 1300.

FIG. 7B may depict steps in a method of mitigating dissociation of a medical device from a patient. The method shown in FIG. 7B may be method 710. In some embodiments, method 710 may comprise the steps of: step 711, step 701, step 705, and step 707. In some embodiments, step 711 may be a step of passing a portion of the medical device through a mitten. In some embodiments, the region of the mitten that is receiving the portion of the medical device may be a tubing-hole 135, e.g., as shown in FIG. 1A. In some embodiments, step 701, step 705, and step 707 of method 710, may be as described above under method 700; but these steps may have a different order in method 710, as shown in FIG. 7B.

In some embodiments, method 700 and/or method 710 may be a method of using a patient-mitten (such as, but not limited to, mittens 100, 200, 300, 400, 500, 600, 800, 1300 combinations thereof, and/or the like) with a medical device. In some embodiments, method 700 and/or method 710 may comprise steps of: step 701 and step 705. In some embodiments, step 701 may be a step of attaching the medical device to at least a region of a limb of a patient. In some embodiments, step 705 may be a step of securing the patient-mitten onto the limb. In some embodiments, after the step 705, the method 700/710 may further comprise step 707 of anchoring a portion of the medical device to the patient-mitten, which may minimize the medical device from becoming unintentionally dissociated from the at least the region of the limb of the patient. See e.g., FIG. 7A and FIG. 7B.

In some embodiments, prior to the step 705, the method 700 may further comprise step 703 of passing a portion of the medical device through the patient-mitten via a passage means of the patient-mitten. In some embodiments, this passage means may be selected from one or more of: access-slit 107, tubing-hole 135, combinations thereof, and/or the like. See e.g., FIG. 7A.

In some embodiments, prior to the step 701, the method 710 may further comprise step 711 of passing a portion of the medical device through the patient-mitten via a passage means of the patient-mitten. In some embodiments, this passage means may be selected from one or more of: access-slit 107, tubing-hole 135, combinations thereof, and/or the like. See e.g., FIG. 7B.

In some embodiments, the at least the region of the limb of the patient with respect to method 700/710 may be selected from one or more of: a finger, a thumb, a hand, a palm, a wrist, or a forearm of that limb.

In some embodiments, the mitten noted in method 710 may be patient-mitten 100, mitten-with-attachment-anchors 200, mitten-with-pocket 300, UV-permeable-mitten 400, mitten-for-protecting-skin-treatment 500, mitten-with-finger-opening 600, mitten 800, patient-mitten 1300, combinations thereof, and/or the like.

FIG. 7C may depict steps in a method of protecting treated skin of a limb. The method shown in FIG. 7C may be method 720. In some embodiments, method 720 may comprise the steps of: step 721, step 723, step 725, and step 727. In some embodiments, step 721 may be a step of applying medicament to skin of a limb; such as skin of the finger, fingers, hand, and/or portions of the arm. In some embodiments, step 723 may be a step of pulling “inner-elongate-sleeve 504” over the limb with medicament. Upon completion of step 723, the mitten may look like the mitten shown in FIG. 5A or FIG. 5B, i.e., with inner-elongate-sleeve 504 deployed/extended over the forearm and elbow. In some embodiments, step 725 may be a step of deploying “outer-elongate-sleeve 503” (e.g., unrolling outer-elongate-sleeve 503) over “inner-elongate-sleeve 504.” Upon completion of step 725, the mitten may look like the mitten shown in FIG. 5C.

In some embodiments, step 727 may be a step of securing “mitten-for-protecting-skin-treatment 500” to the limb with the medicament. In some embodiments, step 727 may involve securing the complimentary fastener-for-arm-grabber 510 of outer-elongate-sleeve 503 to the fastener-for-arm-grabber 510 of inner-elongate-sleeve 504. In some embodiments, step 727 may involve arm-grabber 509 squeezing (gripping) the upper arm of the limb with the medicament.

In some embodiments, method 720 be a method of protecting medicament that has been applied to a skin region of a limb of a patient. In some embodiments, method 720 may comprise steps of: step 721, step 723, step 725, and step 727. In some embodiments, step 721 may be a step of applying the medicament to the skin region. In some embodiments, step 723 may be a step of pulling on an inner layer 504 of a patient-mitten (such as, but not limited to, mittens 100, 200, 300, 400, 500, 600, 800, 1300 combinations thereof, and/or the like) over the skin region, wherein the inner layer 504 may be configured to not absorb the medicament. In some embodiments, step 725 may be a step of pulling on elongate-sleeve 503 of the patient-mitten over at least a portion of the inner layer 504. In some embodiments, step 727 may be a step of securing the patient-mitten to the limb via an arm-grabber 109 of the patient-mitten. In some embodiments, the patient-mitten may comprise hand-cover 101 and elongate-sleeve 103 that may be attached to hand-cover 101. In some embodiments, hand-cover 101 may be configured to removably cover over fingers of the limb. In some embodiments, elongate-sleeve 103 may be configured to removably cover over at least a majority of a forearm of the limb. In some embodiments, elongate-sleeve 103 may terminate in arm-grabber 109 that may be disposed away from hand-cover 101. In some embodiments, the given patient-mitten may comprise the inner layer 504. In some embodiments, when the patient-mitten is being properly and fully worn by the patient, the at least the portion of the inner layer 504 may be disposed beneath the elongate-sleeve 503. In some embodiments, the inner layer 504 may protect the medicament from one or more of: drying out; evaporating; being absorbed into clothing of the patient; being rubbed off from contacts with an environment of the patient; being rubbed off from contact with other parts of the patient; combinations thereof; and/or the like. See e.g., FIG. 7C.

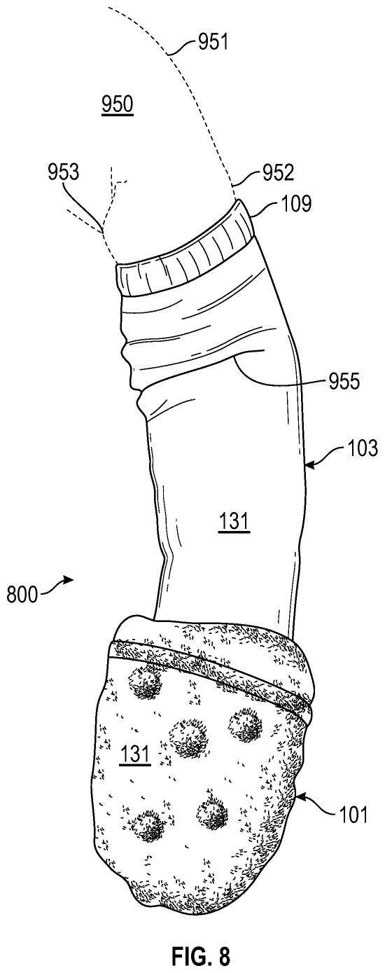

FIG. 8 may depict a patient-mitten 800 being properly worn over a majority of a limb (e.g., a left arm) of a given patient 950. FIG. 8 may show how the arm-grabber 109 of the given patient-mitten 800 may squeeze an upper arm 952 of the patient 950, when the patient-mitten is being properly worn. Shoulder 951 and armpit region 953 of the patient 950 are also shown in FIG. 8 as reference points; as well as elbow region 955. Note when patient-mitten 800 may be properly worn, elbow region 955 may be below arm-grabber 109 that is squeezing upper arm 952.

Note the patient-mitten 800 shown in FIG. 8, may be patient-mitten 100, mitten-with-attachment-anchors 200, mitten-with-pocket 300, UV-permeable-mitten 400, mitten-for-protecting-skin-treatment 500, mitten-with-finger-opening 600, and/or combinations thereof. For example, and without limiting the scope of the present invention, in some embodiments, patient-mitten 800 may comprise: hand-covering 101, elongate-sleeve 103, and arm-grabber 109 as discussed above for patient-mitten 100.

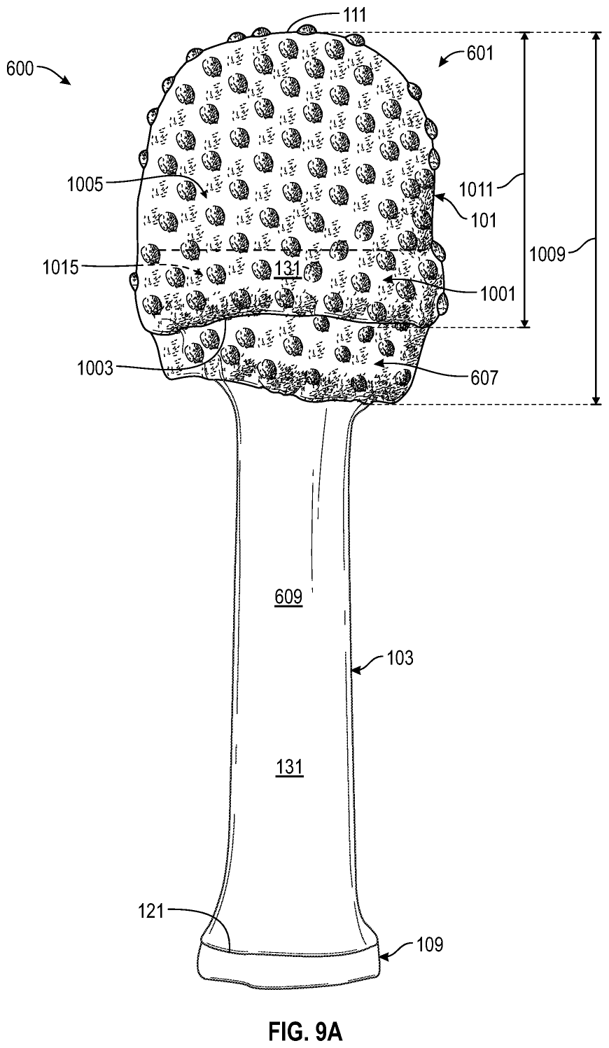

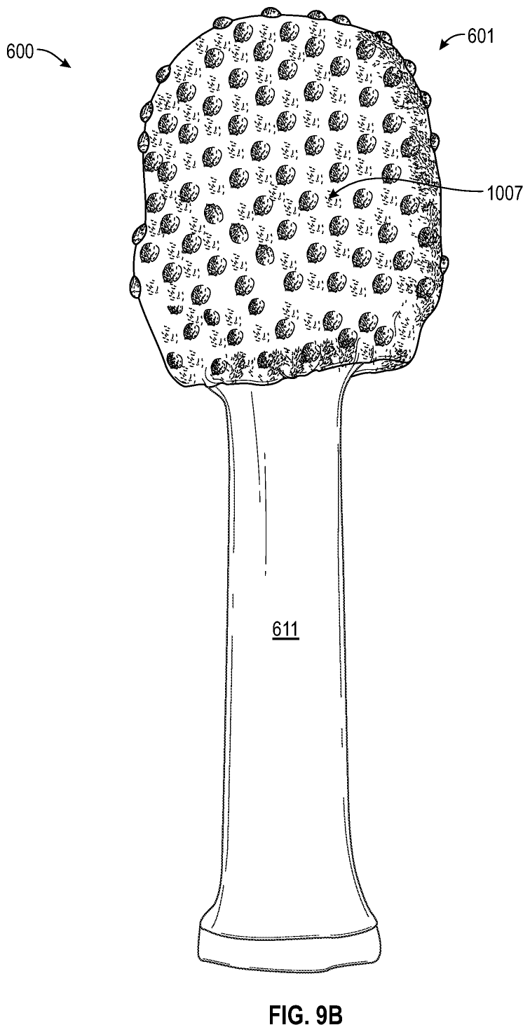

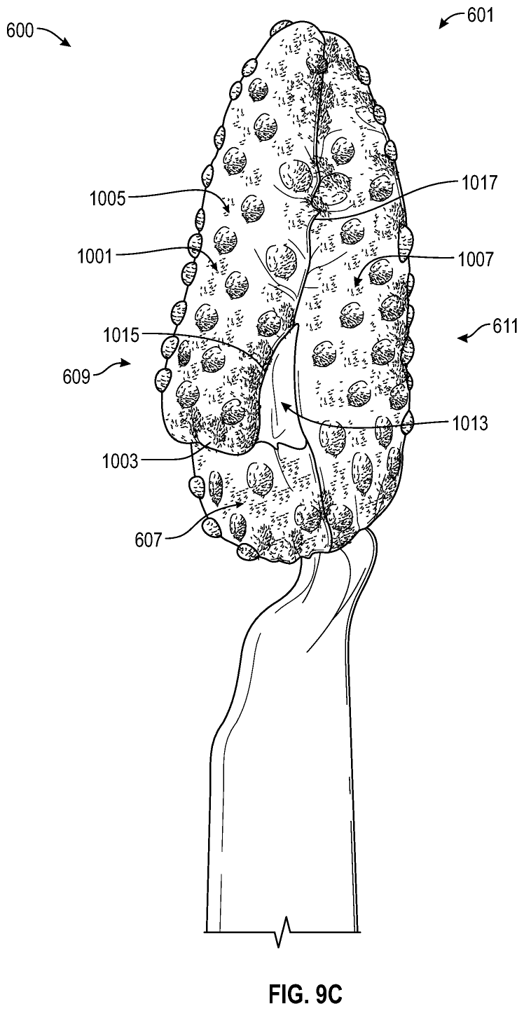

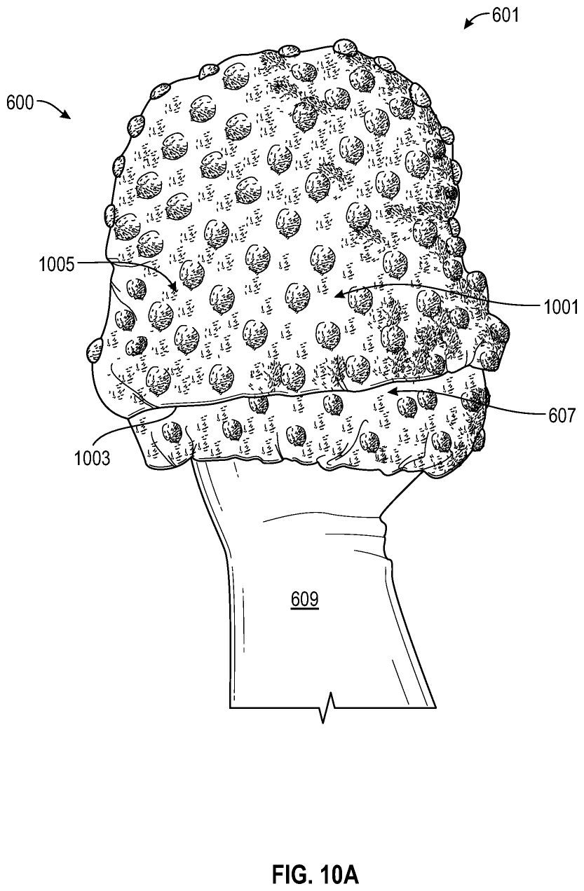

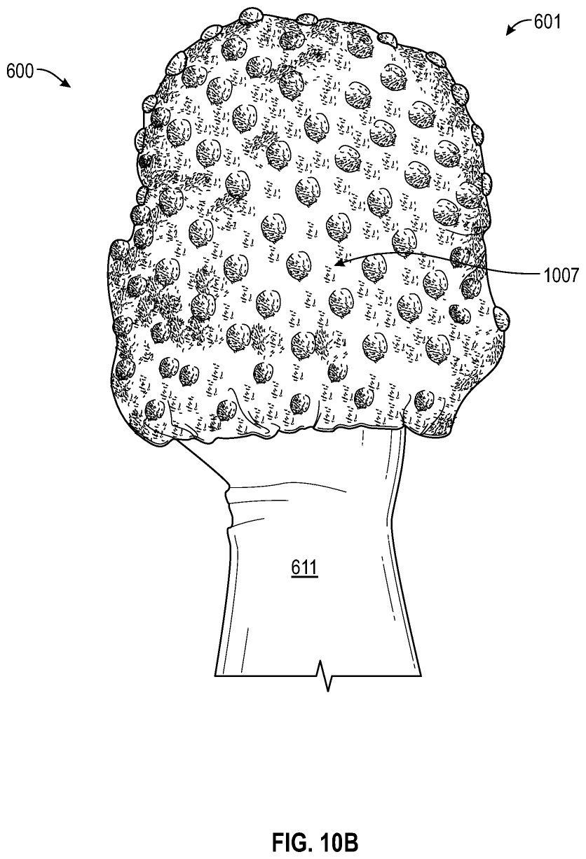

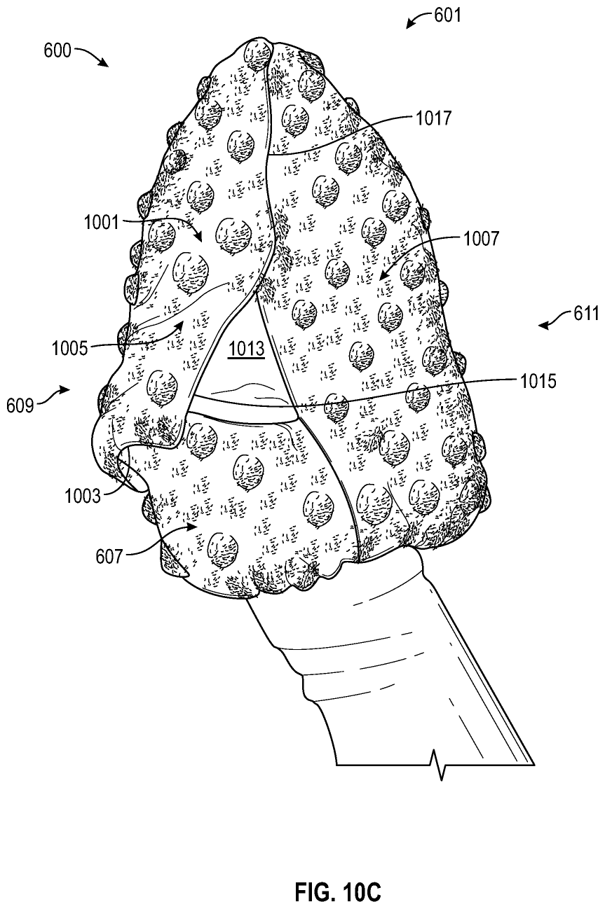

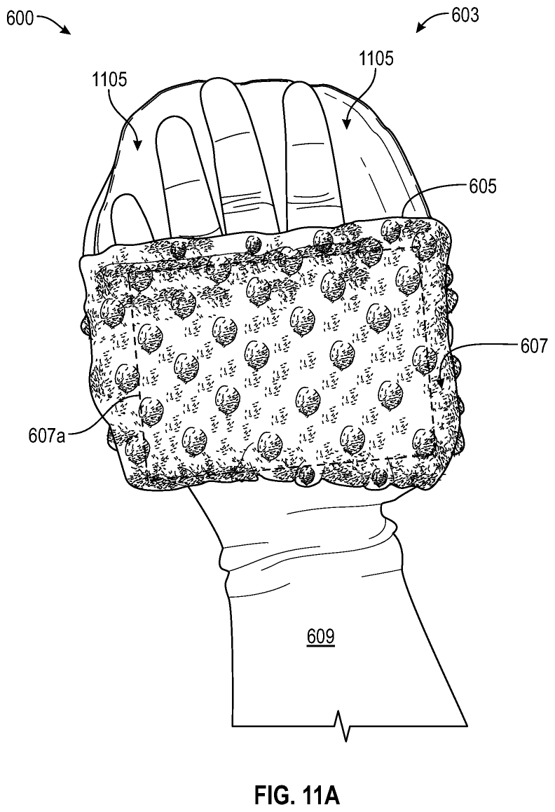

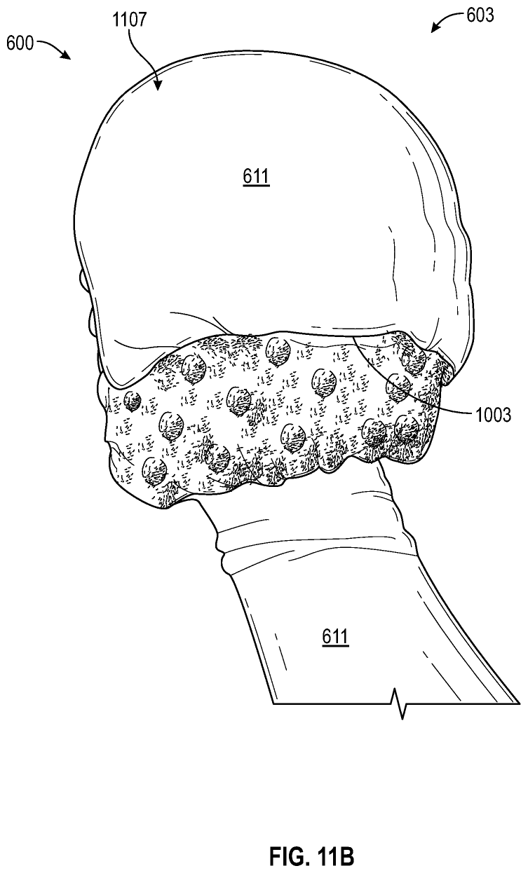

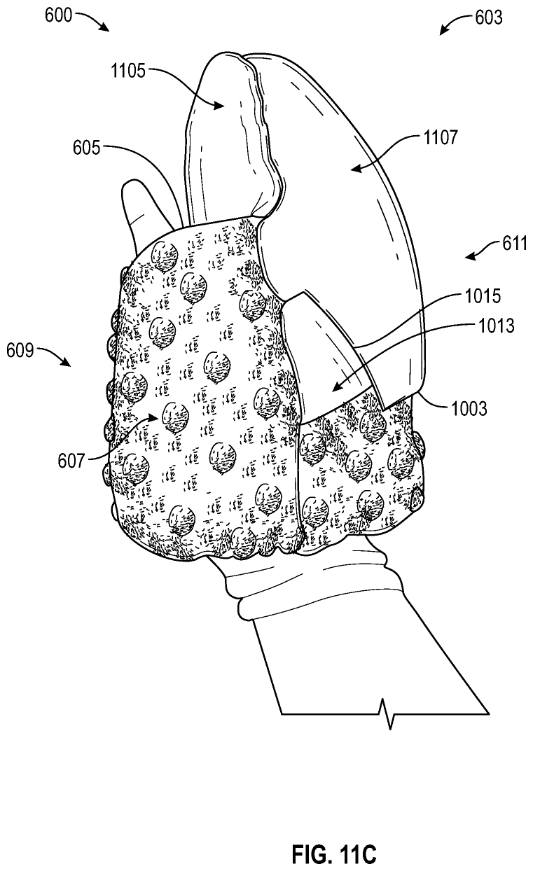

In some embodiments, mitten-with-finger-opening 600 may operationally exist in one of two configurations with respect to whether or not the patient's hand is fully covered or partially covered. In the fully covered configuration 601, all of the patient's hand may be fully covered by hand-covering 101. This fully covered configuration 601 may be shown in FIGS. 9A through 10C. In the partially covered configuration, at least one of patient's finger tips may be uncovered (but the palm of the patient's hand may still be covered). This partially covered configuration may be shown in FIGS. 11A through 11C. Note, in some embodiments, the partially covered configuration (e.g., as shown in FIGS. 11A through 11C) may be substantially the same as uncovered-configuration 603.

FIG. 9A may depict a bottom (ventral) view of mitten-with-finger-opening 600, in its fully covered configuration 601. FIG. 9A may show ventral-side 609 of mitten-with-finger-opening 600. In some, embodiments, ventral-side 609 may be known or denoted as palm-side 609. FIG. 9B may depict a top (dorsal) view of mitten-with-finger-opening 600, in its fully covered configuration 601. FIG. 9B may show dorsal-side 611 of mitten-with-finger-opening 600. In some, embodiments, dorsal-side 611 may be known or denoted as back-of-hand side 611. FIG. 9C may depict a side view of mitten-with-finger-opening 600, in its fully covered configuration 601. Note, in FIGS. 9A through 9C, the mitten-with-finger-opening 600 may be shown without the patient wearing the given mitten-with-finger-opening 600.

FIG. 10A may depict a bottom (ventral) view of mitten-with-finger-opening 600, in its fully covered configuration 601, and while being worn by a patient. FIG. 10A may show ventral-side 609 of mitten-with-finger-opening 600. FIG. 10B may depict a top (dorsal) view of mitten-with-finger-opening 600, in its fully covered configuration 601, and while being worn. FIG. 10B may show dorsal-side 611 of mitten-with-finger-opening 600. FIG. 10C may depict a side view of mitten-with-finger-opening 600, in its fully covered configuration 601, and while being worn.

FIG. 11A may depict a bottom (ventral) view of mitten-with-finger-opening 600, in its partially covered configuration, and while being worn by a patient. FIG. 11B may depict a top (dorsal) view of mitten-with-finger-opening 600, in its partially covered configuration, and while being worn by a patient. FIG. 11C may depict a side view of mitten-with-finger-opening 600, in its partially covered configuration, and while being worn by a patient.

In some embodiments, mitten-with-finger-opening 600 may comprise hand-covering 101 and elongate-sleeve 103 attached and extending from hand-covering 101. In some embodiments, hand-covering 101 may comprise cover-flap 607 and flappable-cover 1001. In some embodiments, when mitten-with-finger-opening 600 may be in the fully covered configuration 601, flappable-cover 1001 may partially cover over cover-flap 607, as shown in FIGS. 9A, 9C, 10A, and 10C.

In some embodiments, when mitten-with-finger-opening 600 may be in the partially covered configuration, flappable-cover 1001 may be flipped over from its position in the fully covered configuration 601, such that flappable-cover 1001 is now not covering cover-flap 607 at all, as shown in FIGS. 11A through 11C.

In some embodiments, when mitten-with-finger-opening 600 may be in the fully covered configuration 601, hand-covering 101 may have the following exterior surfaces: a lower portion of cover-flap 607 (see e.g., FIG. 9A), first-side 1005 of flappable-cover 1001 (see e.g., FIG. 9A), and second-side 1007 of hand-covering 101 (see e.g., FIG. 9B). In some embodiments, first-side 1005 may cover over most of cover-flap 607, aside from the lower portion of cover-flap 607, when mitten-with-finger-opening 600 may be in the fully covered configuration 601 (see e.g., FIG. 9A). In some embodiments, second-side 1007 may be disposed opposite from first-side 1005; i.e., when mitten-with-finger-opening 600 may be in the fully covered configuration 601, first-side 1005 and second-side 1007 may exteriorly face opposite directions from each other (compare FIG. 9A against FIG. 9B). In some embodiments, first-side 1005 may be separated from second-side 1007 by seam 1017 (see e.g., FIG. 9C and FIG. 10C for portions of seam 1017). In some embodiments, seam 1017 may run around the peripheral edge of hand-covering 101, when mitten-with-finger-opening 600 may be in the fully covered configuration. See e.g., FIGS. 9A through 10C.

In some embodiments, when mitten-with-finger-opening 600 may be in the partially covered configuration, hand-covering 101 may have the following exterior surfaces: all of cover-flap 607 (see e.g., FIG. 11A), third-side 1105 of hand-covering 101 (see e.g., FIG. 11A), and fourth-side 1107 of flappable-cover 1001 (see e.g., FIG. 11B). In some embodiments, cover-flap 607 and third-side 1105 may exteriorly face a same direction. In some embodiments, fourth-side 1107 may exteriorly face the opposite direction from cover-flap 607 and third-side 1105. See e.g., FIGS. 11A through 11C. In some embodiments, when mitten-with-finger-opening 600 may be in the partially covered configuration, palm-support 607a (see e.g., FIG. 6A) may support the palm of the wearer; and/or palm-support 607a may prevent the wearer's hand/palm from curling in on itself in an undesired fashion.

Note, when mitten-with-finger-opening 600 may be in the fully covered configuration 601, third-side 1105 and fourth-side 1107 may not be exteriorly facing surfaces; but rather, may be interiorly facing surfaces, facing each other. See e.g., FIG. 9A through FIG. 10C.

Note, first-side 1005 and fourth-side 1107 may be opposite sides of flappable-cover 1001.

Note, second-side 1007 and third-side 1105 may be opposite sides of a portion of hand-covering 101.

In some embodiments, a length-of-hand-covering 1009 may be longer than a length-of-flippable-cover 1011. In some embodiments, length-of-hand-covering 1009 may run from finger-tip-end 111 to wrist-end 113 (or to wrist-union 105 or to distal-end 123). In some embodiments, length-of-flippable-cover 1011 may run from finger-tip-end 111 to flappable-cover-end 1003. In some embodiments, flappable-cover-end 1003 may be where flappable-cover 1001 ends or terminates disposed away from finger-tip-end 111. See e.g., FIG. 9A.

In some embodiments, hand-covering 101 may comprise elastic-strap 1013. In some embodiments, flappable-cover 1001 may comprise elastic-strap 1013. In some embodiments, elastic-strap 1013 may be a predetermined length of elastic strap (or the like), that may run horizontally (width wise) across a bottom portion of flappable-cover 1001. In some embodiments, elastic-strap 1013 may run in a direction that may be substantially (mostly) perpendicular with respect to length-of-hand-covering 1009 (or length-of-flippable-cover 1011). For example, and without limiting the scope of the present invention, when mitten-with-finger-opening 600 may be worn by the patient, elastic-strap 1013 may be substantially (mostly) perpendicular with respect to a length of the patient's arm that is wearing mitten-with-finger-opening 600. In some embodiments, elastic-strap 1013 may attach to opposite portions of seam 1017. In some embodiments, at or proximate (e.g., within a quarter inch) to flappable-cover-end 1003, may be housing-for-elastic 1015, wherein housing-for-elastic 1015 may house most of elastic-strap 1013. In some embodiments, housing-for-elastic 1015 may be a tunnel, open at both of its opposing ends, made at least mostly of fabric. In some embodiments, most of elastic-strap 1013 may run through housing-for-elastic 1015. In some embodiments, hand-covering 101 may comprise housing-for-elastic 1015. In some embodiments, flappable-cover 1001 may comprise housing-for-elastic 1015. In some embodiments, the broken line shown in FIG. 9A that runs across a width hand-covering 101 may indicate an interior boundary of housing-for-elastic 1015. Also see FIGS. 9A and 9C.

In some embodiments, elastic-strap 1013 along with its anchoring to flappable-cover 1001 (via at least in part by housing-for-elastic 1015) and its anchoring to seam 1017, may help to facilitate flipping back and forth between the fully covered configuration 601 and the partially covered configuration. In some embodiments, elastic-strap 1013 along with its anchoring to flappable-cover 1001 (via at least in part by housing-for-elastic 1015) and its anchoring to seam 1017, may help to maintain the overall shape and structural integrity of mitten-with-finger-opening 600 during flipping back and forth between the fully covered configuration 601 and the partially covered configuration. Further, in some embodiments, the generous amount plush material utilized in hand-covering 101, as shown in FIG. 8 through FIG. 11C, may facilitate a greater ease of transitioning between the fully covered configuration and the partially covered configuration (and vice versa), as this generous thickness of plush material provides something of substance to flip over.

In some embodiments, the mittens described and discussed herein (such as, mittens 100, 200, 300, 400, 500, 600, 800, combinations thereof, and/or the like) may be configured for use on a hand and on an arm of a human. In some embodiments, a given such mitten may comprise hand-cover 101 and elongate-sleeve 103. In some embodiments, hand-cover 101 may comprise first-side 1005, second-side 1007, third-side 1105, and fourth-side 1107. In some embodiments, during intended use by the human, when the given mitten is being worn by the human, hand-cover 101 may exist in one of two operational configurations, fully-covered-configuration 601 or uncovered-configuration 603, respectively.

In some embodiments with respect to such mittens, in the fully-covered-configuration 601, the first-side 1005 may form a ventral exterior surface of the hand-cover 101, the second-side 1007 may form a dorsal exterior surface of the hand-cover 101 that opposes at least a majority of the first-side 1005, the first-side 1005 may transition into the second-side 1007 at a first terminal end (e.g., finger-tip-end 111) of the hand-cover 101, the third-side 1105 may form a dorsal interior surface of the hand cover 101 configured to face a dorsal side of at least one finger of the hand, and the fourth-side 1107 may form a ventral interior surface of the hand cover 101 configured to face a ventral side of the at least one finger of the hand. See e.g., FIG. 9A through FIG. 10C.

In some embodiments with respect to such mittens, in the uncovered-configuration 603, the first-side 1005 and the second-side 1007 may form interior surfaces of the hand-cover 101 that face each other without a portion of the human disposed therein, the third-side 1105 may form a different ventral exterior surface of the hand-cover 101 that is still configured to face the dorsal side of the at least one finger of the hand, and the fourth-side 1107 may form a different dorsal exterior surface that substantially faces away from the dorsal side of the at least one finger of the hand. See e.g., FIG. 11A through FIG. 11C.

In some embodiments, the elongate-sleeve 103 that may be substantially hollow and configured for removably covering over at least a portion of the arm of the human/patient. In some embodiments, the elongate-sleeve 103 may be attached to the hand-cover 101. In some embodiments, disposed away from the first terminal end (finger-tip-end 111), the elongate-sleeve 103 may terminate at a second terminal end (e.g., at arm-grabber 109), wherein arm-grabber 109 is located at or proximate (e.g., within one inch) to the second terminal end, wherein the arm-grabber 109 may be configured to removably grip around a circumference of a portion of the arm (e.g., an upper arm portion in some embodiments) of the patient/human.

In some embodiments, a majority of the first-side 1005 may be disposed opposite from a majority of the fourth-side 1107 in both the fully-covered-configuration 601 and in the uncovered-configuration 603. In figures that show first-side 1005, then fourth-side 1107 may be internal within hand-cover 101; whereas, in figures that show fourth-side 1107, then first-side 1005 may be internal within hand-cover 101.

In some embodiments, the first-side 1005 and the fourth-side 1107 form flappable-cover 1001 that is invertible and flappable, to an opposite side of the hand-cover 101, when transitioning from the fully-covered-configuration 601 to the uncovered-configuration 603. For example, compare FIG. 9A through FIG. 10C to FIG. 11A through FIG. 11C. In some embodiments, flappable-cover 1001 may terminate in a flappable-cover-end 1003 that may define a transition from the first-side 1005 to the fourth-side 1107, wherein the flappable-cover-end 1003 is on a ventral side of the hand-cover 101 in the fully-covered-configuration 601 (see e.g., FIG. 10A), wherein the flappable-cover-end 1003 is on a dorsal side of the hand-cover 101 in the uncovered-configuration 603 (see e.g., FIG. 11B).

In some embodiments, the hand-cover 101 may comprise at least one section of an elastic-strap 1013, wherein the at least one section of the elastic-strap 1013 may be attached to at least a portion of the hand-cover 101 and may be in physical communication with the flappable-cover 1001, such that there is at least some elastic resistance between the flappable-cover 1001 and the at least the portion of the hander-cover 101. See e.g., FIG. 10C and FIG. 11C. In some embodiments, hand-cover 101 may comprise housing 1015 that may be configured for housing at least a portion of the at least one section of the elastic-strap 1013, wherein the housing 1015 may be part of flappable-cover 1001. See e.g., FIG. 10C.

In some embodiments, a majority of the second-side 1007 may be disposed opposite from a majority of the third-side 1105 in both the fully-covered-configuration 601 and in the uncovered-configuration 603. In figures that show second-side 1007, then third-side 1105 may be internal within hand-cover 101; whereas, in figures that show third-side 1105, then second-side 1007 may be internal within hand-cover 101.

In some embodiments, hand-cover 101 may comprise flap-cover 607, wherein during the intended use by the human, when the mitten is being worn by the human, flap-cover 607 may be configured to cover over and face at least a portion of the palm. In some embodiments, flap-cover 607 at an end disposed away from the elongate-sleeve 103 may comprise an opening (e.g., opening-for-fingers 605) configured to permit passage of the at least one finger. In some embodiments, in the fully-covered-configuration, the fourth-side 1107 (and the first-side 1005) may cover over most of flap-cover 607 (see e.g., FIG. 9A and FIG. 10A). In some embodiments, in the uncovered-configuration 603 the flap-cover 607 is not covered over by the fourth-side 1107 (see e.g., FIG. 11C).

In some embodiments, hand-cover 101 may comprise palm-cover 607a. Palm-cover 607a may be shown in some drawing figures in broken lines to indicate that 607a may be disposed between exterior/interior facing surfaces/fabrics/coverings or the like. In some embodiments, palm-cover 607a may be disposed within (or beneath) flap-cover 607. In some embodiments, palm-cover 607a may have a minimal rigidity to minimize unintentional curling of the palm (and/or of the fingers), wherein palm-cover 607a may be configured to minimize the unintentional curling of the palm. In some embodiments, palm-cover 607a may be more firm, rigid, and/or dense than other regions/portions of a given mitten. See e.g., FIG. 6A and FIG. 11A.

In some embodiments, at least some fabric(s) of hand-cover 101 may be twice as much or more thick as compared to at least some fabric(s) of elongate-sleeve 103.

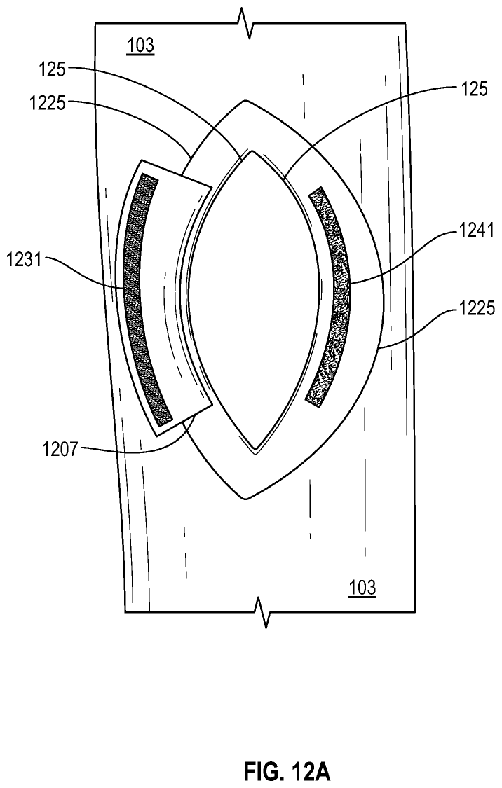

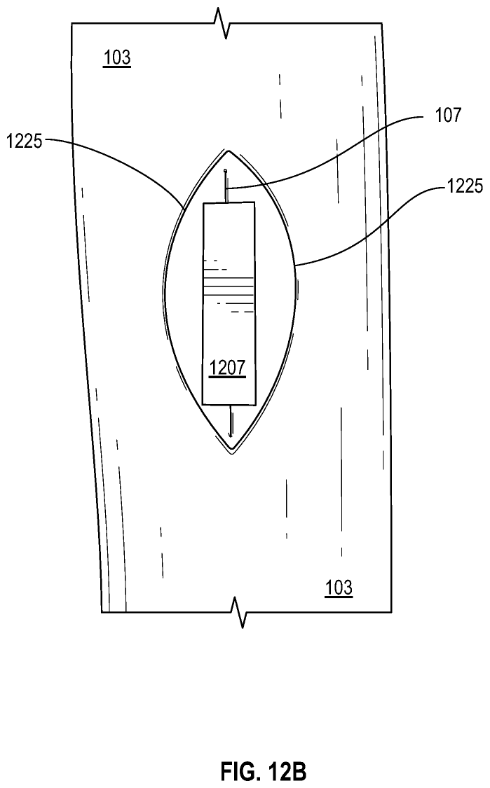

FIG. 12A and FIG. 12B may show portions of elongate-sleeve 103 with at least one access-slit 107. In FIG. 12A access-slit 107 may be removably open. In FIG. 12B access-slit 107 may be removably closed (sealed). In some embodiments, around access-slit 107 on elongate-sleeve 103 may be a predetermined and/or defined region 1225. In some embodiments, region 1225 may be a region of reinforced stitching. In some embodiments, access-slit 107 may be located within region 1225. In some embodiments, access-slit 107 may be a slit that may run mostly (substantially) down a length of region 1225. In some embodiments, opposites sides of access-slit 107, denoted as complimentary-edges 125, may form different sides of region 1225. In some embodiments, on one side of region 1225 may be flap 1207. In some embodiments, flap 1207 may extend from one side of region 1225. In some embodiments, flap 1207 may be a flap of material, such as, but not limited to fabric/textile. In some embodiments, running along an underside of flap 1207 may be a fastener 1231. In some embodiments, on the opposite side of region 1225, that flap 1207 does not extend from, may be a complimentary-fastener 1241. In some embodiments, fastener 1231 and complimentary-fastener 1241 may removably and complimentary attach to each other. In some embodiments, fastener 1231 and complimentary-fastener 1241 may be one or more mechanical fasteners. In some embodiments, fastener 1231 and complimentary-fastener 1241 may one or more of: plurality of hooks and plurality loops connectors (e.g., Velcro or the like), buttons, snaps, ties, zippers, ziplock, and/or the like. In some embodiments, access-slit 107 may run substantially a same length as elongate-sleeve 103. In some embodiments, access-slit 107 may have length that is less than a length of elongate-sleeve 103.

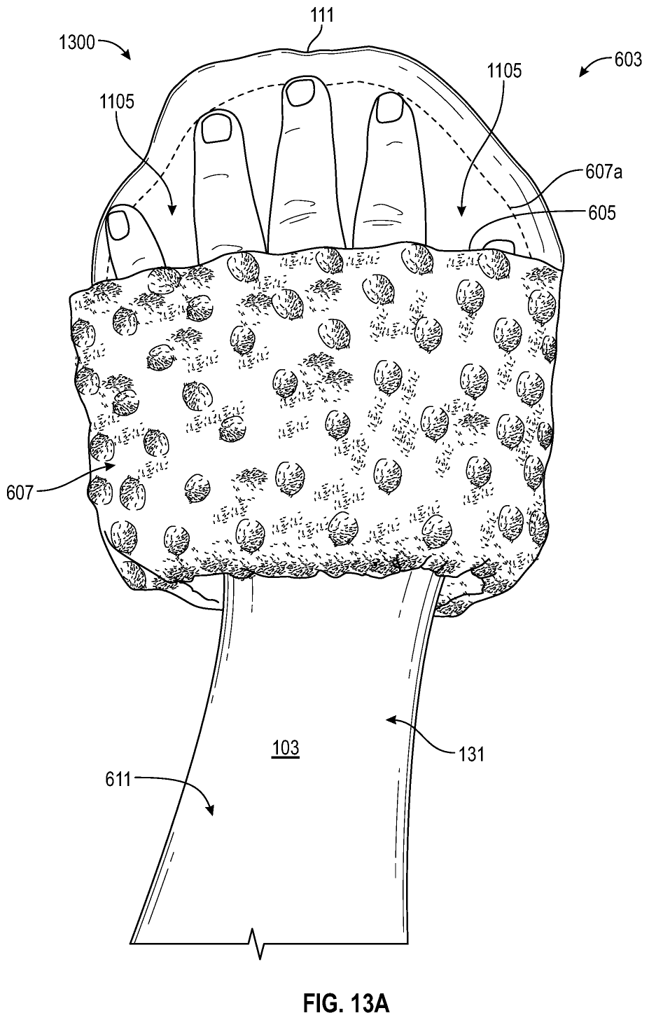

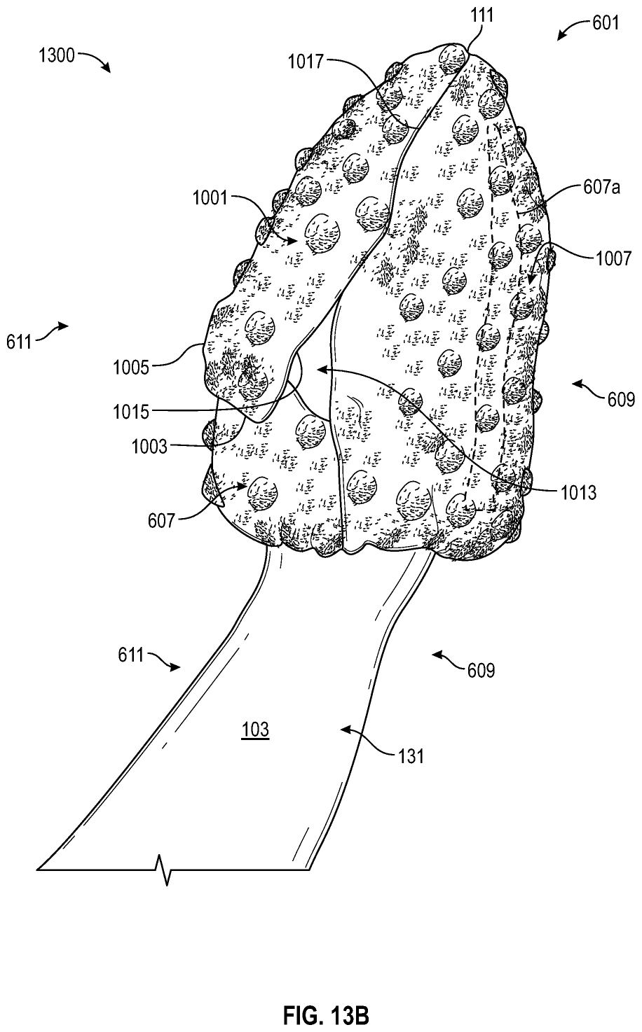

FIG. 13A may depict a top (dorsal) view of a mitten-with-finger-opening (patient-mitten 1300), in its partially covered configuration, while being worn, showing at least some fingernails of the patient's hand visible. In contrast, FIG. 13B may depict a side view of the mitten-with-finger-opening (patient-mitten 1300) of FIG. 13A, in its fully covered configuration, and while being worn (with no part of the patient's hand visible). In some embodiments, patient-mitten 1300 may be substantially similar to mitten-with-finger-opening 600 but with at least two differences, (1) the dorsal-ventral positions may be reversed in patient-mitten 1300 as compared to mitten-with-finger-opening 600; and (2) palm-support 607a in patient-mitten 1300 may be disposed between third-side 1105 and second-side 1007; otherwise, patient-mitten 1300 may be constructed in a substantially similar manner/fashion as that of mitten-with-finger-opening 600. In some embodiments of patient-mitten 1300, a bottom/ventral side of the patient's hand (e.g., the palm side) may be supported and/or rest against third-side 1105, in either of the fully-covered-configuration 601 (e.g., FIG. 13B) or in the uncovered-configuration 603 (e.g., FIG. 13A); thus, in the dorsal/top view of FIG. 13A at least some of the patient's fingernails may be visible; whereas, in comparison in FIG. 11A, in some embodiments, of mitten-with-finger-opening 600, the patient's hand is reversed within mitten-with-finger-opening 600, such that no fingernails of the patient are visible from the bottom/ventral view of FIG. 11A (because the top/dorsal part of the patient's hand is against third-side 1105 and the ventral/bottom/palm side is against cover-flap 607). In some embodiments of patient-mitten 1300, palm-support 607a in patient-mitten 1300 may be disposed between third-side 1105 and second-side 1007 so that palm-support 607a may provide at least some support and/or rigidity to third-side 1105; and a bottom/ventral side of the patient's hand (e.g., the palm side) may then be supported and/or rest against third-side 1105 (with internal palm-support 607a); wherein this arrangement may prevent or minimize the palm and/or the fingers of the patient from curling in on themselves. In some embodiments, palm-support 607a may be more rigid, firm, and/or dense than other regions/portions of the given mitten (e.g., patient-mitten 1300). In some embodiments, palm-support 607a may be more rigid, firm, and/or dense than other regions/portions of the given mitten (e.g., patient-mitten 1300) that are proximate and/or that are in communication with palm-support 607a. In some embodiments, palm-support 607a may be substantially to entirely surrounded by other material(s) that are less firm, rigid, and/or dense as compared to palm-support 607a.

Note, some embodiments of patient-mittens and/or of mitten-with-finger-opening 600 may or may not comprise one or more palm-supports 607a. Some embodiments of patient-mittens and/or of mitten-with-finger-opening 600 may comprise one or more palm-supports 607a (e.g., within cover-flap 607 and/or disposed between third-side 1105 and second-side 1007). Some embodiments of patient-mittens and/or of mitten-with-finger-opening 600 may not have any palm-supports 607a.

Note, any of the structures and/or features noted in patient-mitten 100, mitten-with-attachment-anchors 200, mitten-with-pocket 300, UV-permeable-mitten 400, mitten-for-protecting-skin-treatment 500, mitten-with-finger-opening 600, patient-mitten 800, and/or patient-mitten 1300 may be used and/or incorporated in the various mittens disclosed herein. For example, and without limiting the scope of the present invention, one or more of tubing-hole(s) 135, access-slit(s) 107, attachment-anchor(s) 201, pocket(s) 301, and/or the like may be used and/or incorporated in mitten-with-finger-opening 600. For example, and without limiting the scope of the present invention, the ability to convert from the fully covered configuration into the partially covered configuration, and vice-versa, of mitten-with-finger-opening 600, may be applied and/or incorporated into patient-mitten 100, mitten-with-attachment-anchors 200, mitten-with-pocket 300, UV-permeable-mitten 400, mitten-for-protecting-skin-treatment 500, patient-mitten 800, or patient-mitten 1300.

As noted above, various aspects of the discussed mittens and patient-mittens may have soft and/or flexible portions. For example, and without limiting the scope of the present invention, substantial portions of the following structures may be substantially constructed from fabrics: hand-covering 101, elongate-sleeve 103, exterior 131, interior surface 137, pocket 301, outer-elongate-sleeve 503, inner-elongate-sleeve 504, cover-flap 607, flappable-cover 1001, flappable-cover-end 1003, first-side 1005, second-side 1007, elastic-strap 1013, housing-for-elastic 1015, third-side 1105, fourth-side 1107, and/or the like. And such fabrics may be soft and/or flexible, as one of ordinary skill in the textiles arts would understand softness and flexibility. For example, and without limiting the scope of the present invention, such fabrics may be natural (such as, but not limited to, cotton, silk, hemp, linen, combinations thereof, and/or the like), synthetic (such as, but not limited to, polyester, nylon, acrylic, acetate, Rayon, Viscose, Spandex, combinations thereof, and/or the like), combinations thereof, and/or the like.

In some embodiments use of fabric noted herein in and on various mittens and/or patient-mittens may be natural fabrics, synthetic fabrics, combinations thereof, and/or the like. Such natural fabrics may be one or more of: cotton, silk, linen, hemp, wool, cashmere, leather, combinations thereof, and/or the like. Such synthetic fabrics may be one or more of: polyester, nylon, acrylic, acetate, Rayon, Viscose, Spandex, Gore-Tex, combinations thereof, and/or the like. And as noted above, such fabrics, or portions thereof, may also be UV permeable fabrics, liquid impermeable fabrics (waterproof fabrics) and/or air breathable fabrics.

Additionally, note mittens utilizing air breathable embodiments of the various elongate-sleeves (terminating in an arm-grabber), may be utilized in activities that might produce sweat, but where it may be desirable for one to cover portions of their arm, hand, and/or fingers during such activity; such as, but not limited to, runners, athletes, and/or the like. At either end of such a mitten, may be attachment hardware (e.g., a clip) for removable attachment of the given mitten to an article, such as, but not limited to a belt. Similarly, such mittens utilizing air breathable embodiments of the various elongate-sleeves (terminating in an arm-grabber), may be utilized in hot or warm environmental conditions.

In some embodiments, within fabrics of the mittens, may be one or more inner materials, such as, but not limited to, rigid to semi-rigid materials, plastic(s), batting, foam, plush, thicker plush, denser plush, regions of thicker plush, regions of denser plush, thicker batting, denser batting, regions of thicker batting, regions of denser batting, thicker foam, denser foam, regions of thicker foam, regions of denser foam, thicker fabric, denser fabric, regions of thicker fabric, regions of denser fabric, combinations thereof, and/or the like. In some embodiments, palm-support 607a may be such an inner material. For example, and without limiting the scope of the present invention, in some embodiments, palm-support 607a may be a region of denser/thicker plush material (or the like) as compared to surrounding materials.

In some embodiments, within fabrics of the mittens, may be one or more rigid to semi-rigid materials, such as, but not limited, to: plastic, thermoplastic, foam, open celled foam, closed cell foam, polyethylene foam, polyvinylchloride (PVC) foam, ethylene-vinyl acetate (EVA) foam, memory foam, wood, metal, cardboard, laminates, composites, combinations thereof, and/or the like. In some embodiments, these rigid to semi-rigid materials may be used in the mittens to help prevent (or to help minimize) the fingers of a patient's hand from curling in towards the palm of the hand in an undesired manner. That is, these rigid to semi-rigid materials may function as rigid to semi-rigid scaffolding or a splint like structure with respect to the patient's hand and/or fingers. That is, these rigid to semi-rigid materials may form a majority of palm-support 607a. These rigid to semi-rigid materials may be more rigid that the fabric(s) that may cover over these rigid to semi-rigid materials. For example, if a given length of cotton fabric is stood up on end, it will merely collapse (fold) back down on itself; whereas, these rigid to semi-rigid materials could remain erect because of their inherent rigidity. For example, and without limiting the scope of the present invention, hand-covering 101, cover-flap 607, palm-support 607a, flippable-cover 1001, first-side 1005, second-side 1007, third-side 1105, fourth-side 1107, combinations thereof, and/or the like may contain or may be comprised of such one or more rigid to semi-rigid materials (e.g., within their fabric(s) covering(s)). In some embodiments, these one or more rigid to semi-rigid materials may be thin elongate members, such as, but not limited to, planar, plate, or disc in shape. In some embodiments, these one or more rigid to semi-rigid materials may be a region of denser/thicker plush material, batting, foam, combinations thereof, and/or the like. For example, and without limiting the scope of the present invention, in some embodiments, pal-support 607a may be comprised of such one or more rigid to semi-rigid materials.

Note with respect to the materials of construction, it is not desired nor intended to thereby unnecessarily limit the present invention by reason of such disclosure.

Various types of mittens, patient-mittens, and methods of use that utilize such mittens have been disclosed and described. The foregoing description of the various exemplary embodiments of the invention has been presented for the purposes of illustration and disclosure. It is not intended to be exhaustive or to limit the invention to the precise form disclosed. Many modifications and variations are possible in light of the above teaching without departing from the spirit of the invention.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A mitten configured for use on a hand and on an arm of a human, wherein the mitten comprises:
a hand-cover comprising a first-side, a second-side, a third-side, and a fourth-side; wherein during intended use by the human, when the mitten is being worn by the human, the hand-cover exists in two operational configurations, a fully covered configuration and an uncovered configuration, respectively;
wherein in the fully covered configuration, the first-side forms a ventral exterior surface of the hand-cover, the second-side forms a dorsal exterior surface of the hand-cover that opposes at least a majority of the first-side, the first-side transitions into the second-side at a first terminal end of the hand-cover, the third-side forms a dorsal interior surface of the hand cover configured to face a dorsal side of at least one finger of the hand, and the fourth-side forms a ventral interior surface of the hand cover configured to face a ventral side of the at least one finger of the hand;
wherein in the uncovered configuration, the first-side and the second-side form interior surfaces of the hand-cover that face each other without a portion of the human disposed therein, the third-side forms a different ventral exterior surface of the hand-cover that is still configured to face the dorsal side of the at least one finger of the hand, the fourth-side forms a different dorsal exterior surface that substantially faces away from the dorsal side of the at least one finger of the hand; and
an elongate-sleeve that is substantially hollow and configured for removably covering over at least a portion of the arm; wherein the elongate-sleeve is attached to the hand-cover; wherein disposed away from the first terminal end, the elongate-sleeve terminates at a second terminal end, wherein an arm-grabber is located at or proximate to the second terminal end, wherein the arm-grabber is configured to removably grip around a circumference of a portion of the arm.

2. The mitten according to claim 1, wherein the majority of the first-side is disposed opposite from a majority of the fourth-side in both the fully covered configuration and in the uncovered configuration.

3. The mitten according to claim 2, wherein the first-side and the fourth-side form a flappable-cover that is inverted and flipped to an opposite side of the hand-cover when transitioning from the fully covered configuration to the uncovered configuration.

4. The mitten according to claim 3, wherein the flappable-cover terminates in a flappable-cover-end that defines a transition from the first-side to the fourth-side, wherein the flappable-cover-end is on a ventral side of the hand-cover in the fully covered configuration, wherein the flappable-cover-end is on a dorsal side of the hand-cover in the uncovered configuration.

5. The mitten according to claim 4, wherein the hand-cover comprises at least one section of an elastic-strap, wherein the at least one section of the elastic-strap is attached to at least a portion of the hand-cover and is in physical communication with the flappable-cover, such that there is at least some elastic resistance between the flappable-cover and the at least the portion of the hander-cover.

6. The mitten according to claim 5, wherein the hand-cover comprises a housing that is configured for housing at least a portion of the at least one section of the elastic-strap, wherein the housing is part of the flappable-cover.

7. The mitten according to claim 1, wherein a majority of the second-side is disposed opposite from a majority of the third-side in both the fully covered configuration and in the uncovered configuration.

8. The mitten according to claim 1, wherein the hand-cover comprises a flap-cover, wherein during the intended use by the human, when the mitten is being worn by the human, the flap-cover is configured to cover over and face at least a portion of the palm; wherein the flap-cover at an end disposed away from the elongate-sleeve comprises an opening configured to permit passage of the at least one finger; wherein in the fully covered configuration, the fourth-side covers over most of the flap-cover; wherein in the uncovered configuration the flap-cover is not covered over by the fourth-side.

9. The mitten according to claim 8, wherein the hand-cover comprises a palm-cover, wherein the palm-cover is disposed within the flap-cover, wherein the palm-cover has a minimal rigidity to minimize unintentional curling of the palm, wherein the palm-cover is configured to minimize the unintentional curling of the palm.

10. A method of using a patient-mitten with a medical device, comprising steps of:
(a) attaching the medical device to at least a region of a limb of a patient; and
(b) securing the patient-mitten onto the limb, wherein the patient-mitten comprises a hand-cover and an elongate-sleeve that is attached to the hand-cover, wherein the hand-cover is configured to removably cover over fingers of the limb, wherein the elongate-sleeve is configured to removably cover over at least a majority of a forearm of the limb.

11. The method according to claim 10, wherein after the step (b), the method further comprises a step of anchoring a portion of the medical device to the patient-mitten which minimizes the medical device from becoming unintentionally dissociated from the at least the region of the limb of the patient.

12. The method according to claim 10, wherein prior to the step (a), the method further comprises a step of passing a portion of the medical device through the patient-mitten via a passage means of the patient-mitten.

13. The method according to claim 12, wherein the passage means is selected from one or more of: an access slit in the patient-mitten or a hole in the patient-mitten.

14. The method according to claim 10, wherein prior to the step (b), the method further comprises a step of passing a portion of the medical device through the patient-mitten via a passage means of the patient-mitten.

15. The method according to claim 14, wherein the passage means is selected from one or more of: an access slit in the patient-mitten or a hole in the patient-mitten.

16. The method according to claim 10, wherein the at least the region of the limb of the patient is selected from one or more of: a finger, a thumb, a hand, a palm, a wrist, or a forearm of that limb.

17. A method of protecting medicament that has been applied to a skin region of a limb of a patient, the method comprising steps of:
(a) applying the medicament to the skin region;
(b) pulling on an inner layer of a patient-mitten over the skin region, wherein the inner layer is configured to not absorb the medicament;
(c) pulling on an elongate-sleeve of the patient-mitten over at least a portion of the inner layer; and
(d) securing the patient-mitten to the limb via an arm-grabber of the patient-mitten, wherein the arm-grabber is configured to removably grip around a circumference of a portion of the limb;
wherein the patient-mitten comprises a hand-cover and the elongate-sleeve that is attached to the hand-cover, wherein the hand-cover is configured to removably cover over fingers of the limb, wherein the elongate-sleeve is configured to removably cover over at least a majority of a forearm of the limb, wherein the elongate-sleeve terminates in the arm-grabber disposed away from the hand-cover, wherein the patient-mitten comprises the inner layer, wherein when the patient-mitten is being properly and fully worn by the patient, the at least the portion of the inner layer is disposed beneath the elongate-sleeve.

18. The method according to claim 17, wherein the inner layer protects the medicament from one or more of: drying out; evaporating; being absorbed into clothing of the patient; being rubbed off from contacts with an environment of the patient; or being rubbed off from contact with other parts of the patient.

19. The method according to claim 17, wherein the medicament is selected from one or more of: a topical medication, a cream, a lotion, a salve, an ointment, a gel, a foam, a film, an oil, an essential oil, an unguent, a balm, or an emulsion.
